(19) United States
(12) Patent Application Publication
(10) Pub. No.: US 2022/0411646 A1
(43) Pub. Date: Dec. 29, 2022

(54) COMPOSITIONS AND METHODS FOR PROTECTING COATINGS FROM THE DELETERIOUS EFFECTS OF EXPOSURE TO UV-C LIGHT

(71) Applicant: CYTEC INDUSTRIES INC., Princeton, NJ (US)

(72) Inventors: Lei ZHANG, Stamford, CT (US); Ram B. GUPTA, Stamford, CT (US); Jian-Yang CHO, Easton, CT (US); Jerry Mon Hei ENG, Wilton, CT (US); Xin LI, Westport, CT (US)

(73) Assignee: CYTEC INDUSTRIES INC., Princeton, NJ (US)

(21) Appl. No.: 17/849,614

(22) Filed: Jun. 25, 2022

Related U.S. Application Data
(60) Provisional application No. 63/216,172, filed on Jun. 29, 2021.

(51) Int. Cl. C09D 5/32; C09D 175/04; C09D 133/08; C09D 7/48; C09D 7/61; C07D 251/14

(52) U.S. Cl. CPC C09D 5/32; C09D 175/04; C09D 133/08; C09D 7/48; C09D 7/61; C07D 251/14

(57) ABSTRACT

Coating compositions having a polymeric binder and a stabilizer composition including at least one of a UV absorber, a hindered amine light stabilizer (HALS), or an inorganic UV blocker are provided herein, wherein said coating compositions are more resistant to discoloration when exposed to UV-C (190-280 nm) light compared to the coating compositions in the absence of the stabilizer composition. Thus, a method of stabilizing a coating composition against the deleterious effects of UV-C (190-280 nm) light includes adding to the coating composition an effective amount of such a stabilizer composition, wherein the coating composition is made more resistant to discoloration when exposed to UV-C (190-280 nm) light compared to the coating composition in the absence of the stabilizer composition. Coated articles can be made from the coating composition, and methods of making stabilized coating films include adding the stabilizer composition to the coating composition.

### CROSS-REFERENCE TO RELATED APPLICATIONS

This application claims benefit of priority to U.S. Provisional Application No. 63/216,172 filed Jun. 29, 2021, the entire content of which is explicitly incorporated herein by reference.

### BACKGROUND

### Field

This disclosure generally relates to protecting coatings from the deleterious effects of exposure to UV-C light and to classes of stabilizers effective in protecting coatings from degradation caused by exposure to UV-C light.

### Description of the Related Art

Most organic materials undergo photodegradation when exposed to ultraviolet (UV) light, leading to irreversible chemical changes. These changes adversely affect the physical properties of the organic material. Various organic materials are utilized as binders in coating compositions, and irreversible chemical changes in the binder caused by UV light can result in discoloration, yellowing, cracking, crazing, gloss loss, hazing (reduced clarity and brilliance), and delamination in coatings.

Ultraviolet (UV) light ranges from 100 to 400 nm, and is classified into three sub-regions, viz., UV-A (320 to 400 nm), UV-B (280 to 320 nm), and (UV-C) 100 to 280 nm. The primary source of UV light is sunlight. Solar light in the UV-C range does not reach Earth's surface as it gets absorbed by the ozone layer of the stratosphere and by oxygen in the layers above. Thus, immense efforts have been made over the past more than 60 years to develop stabilizers to protect organic polymeric materials and human skin from the deleterious effects of UV-B and UV-A light. It is no surprise though that stabilization against UV-C light has largely been ignored, because it was never an issue. However, with the spread of the novel coronavirus COVID-19 the world over, scientists have been endeavoring to reduce transmission in various ways, including disinfecting with UV-C light, which is germicidal. Within a short period of time there has been an exponential growth in utilization of UV-C light as a disinfectant tool, mainly for indoor applications. Various UV-C devices are being fabricated and used for indoor applications, e.g., for disinfection in medical buildings/hospitals; various modes of transportation, such as, airplanes, trains, automobiles, buses (including stations and airports); commercial and residential interiors including retail stores, restaurants, bars; indoor equipment including furniture, paints, personal protective equipment (PPE), carpets and textiles, and electrical and electronic devices, etc. For example, the UV-C dose received in different areas in a hospital intensive care unit ward room after an automated UV-C decontamination was discussed in “Ultraviolet-C decontamination of a hospital room: Amount of UV light needed” (Burns, Issue 46, pp. 842-849).

Indoor furnishings, such as walls, ceilings, furniture, electronic devices, medical devices, decorative items, etc.) generally have surface coatings which are potentially susceptible to degradation by the applied UV-C light. The preferred UV-C wavelength range for disinfection is considered to be between 200 to 280 nm, and the especially preferred range is 222 to 254 nm. It has been demonstrated that UV-C exposure effectively and efficiently inactivates microorganisms, including the COVID-19 virus. Normally, indoor surface coatings are not formulated with UV stabilizers due to the limited exposure to UV-A and UV-B light indoors. However, there appears to be a lack of understanding of what adverse effects UV-C exposure might have on surface coatings. Of particular concern is the fact that UV-C light is of higher energy than UV-A and UV-B light, and can theoretically be more harmful to coatings. For example, as stated in “The Pains of Increased Disinfection Protocols on Wall Coatings in Healthcare” (Paint and Coatings Industry, Oct. 7, 2020), “high-energy UV-C light unit systems are used to disinfect or sterilize surfaces in critical care rooms . . . and, in the case of the UV-C method, cause color changes of certain wall coating types”.

UV stabilizers are likewise susceptible to degradation by UV light. There is a lack of understanding of the effect of UV-C light on stabilizers generally used to protect coatings against UV-A and UV-B light. Stabilizers may be more susceptible to UV-C light than to UV-A or UV-B light, and may degrade and lose their efficacy in protecting coatings upon UV-C exposure more quickly than upon UV-A and UV-B exposure. There has not been much effort in the prior art to address the stabilization of coatings against UV-C light. Thus, there is an urgent need for stabilizer compositions to protect coatings from exposure to UV-C light. The stabilizer compositions should be inherently stable to UV-C light. Moreover, there is a need for stabilizer compositions that impart resistance to discoloration, cracking, crazing, gloss loss, hazing (reduced clarity and brilliance), and delamination to coatings when exposed to UV-C light. Such stabilizer compositions would be a useful advance in the art and could find rapid acceptance by industry.

### SUMMARY

Coating compositions comprising a polymeric binder and a stabilizer composition comprising at least one of a UV absorber, a hindered amine light stabilizer (HALS), or an inorganic UV blocker, is more resistant to discoloration when exposed to UV-C (190-280 nm) light compared to the coating composition in the absence of the stabilizer composition.

Methods of forming a coated article comprise applying a layer of a coating composition comprising a polymeric binder and at least one of a UV absorber, a hindered amine light stabilizer (HALS), or an inorganic UV blocker to a surface of a substrate; and drying or allowing the coating composition to dry by any suitable means.

Methods of making a stabilized coating film comprise: adding an effective amount of a stabilizer composition comprising at least one of a UV absorber, a hindered amine light stabilizer (HALS), or an inorganic UV blocker to a coating composition comprising a polymeric binder; applying a layer of the coating composition to a surface of a substrate; and drying or allowing the coating composition to dry by any suitable means to provide the stabilized coating film.

Methods of stabilizing a coating composition against the deleterious effects of UV-C (190-280 nm) light comprises adding to the coating composition having a polymeric binder an effective amount of a stabilizer composition comprising at least one of a UV absorber, a hindered amine light stabilizer (HALS), or an inorganic UV blocker, wherein the coating composition is more resistant to discoloration when exposed to UV-C (190-280 nm) light compared to the coating composition in the absence of the stabilizer composition.

Use of the coating compositions and/or films described herein serve to protect substrates from the environment and to provide an aesthetic effect. Articles coated with such compositions or films can be classified according to their end-uses. The end-use application can be, for example, transportation coatings, automotive coatings, refinish coatings, general industrial coatings, coil coatings, heavy-duty maintenance coatings, marine coatings, architectural coatings, wood stains and varnishes, paper coatings, or inks.

This summary may not list all characteristics or elements of the coating compositions and methods, and subcombinations of elements may also constitute an embodiment. These and other objects, features, and advantages of the coating compositions and methods will become apparent from the following Detailed Description taken in conjunction with the accompanying Examples section.

### DETAILED DESCRIPTION

The present inventors have discovered that stabilizer compositions comprising at least one of a UV absorber, a hindered amine light stabilizer (HALS), or an inorganic UV blocker are particularly useful for making coating compositions resistant to the deleterious effects of exposure to UV-C light. Advantageously, the coating compositions are more resistant to discoloration when exposed to UV-C (190-280 nm) light compared to the coating compositions in the absence of the stabilizer compositions. The coating compositions can also be more resistant to cracking, crazing, gloss loss, hazing (reduced clarity and brilliance), and delamination when exposed to UV-C (190-280 nm) light compared to the coating compositions in the absence of the stabilizer compositions.

The coating compositions and methods disclosed and/or claimed herein are not intended to encompass any previously known product/compositions, processes for making such products or compositions, or any methods of using the products or compositions. Accordingly, Applicant reserves the right to disclaim any portion of any previously known products, processes, or methods that inadvertently overlap with coating compositions and methods hereby disclosed and/or claimed.

### Definitions

Unless otherwise defined, all terms of art, notations and other scientific or industrial terms or terminology used herein are intended to have the meanings commonly understood by those of skill in the chemical arts. In some cases, terms with commonly understood meanings are defined herein for clarity and/or for ready reference, and the inclusion of such definitions herein should not necessarily be construed to represent a substantial difference over the definition of the term as generally understood in the art unless otherwise indicated.

A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations” is incorporated herein by reference.

The terms “radiation” and “light” when used in the context of UV-A, UV-B, and UV-C are synonymous.

The term “coating composition” refers to a substance that is applied as a liquid, paste, or solid, and can be dried, cured, or dried and cured to form a solid coating film adhering to a substrate. The term “paint” is synonymous with coating composition.

The terms “coating” and “coating film” refer to a layer of a dried or crosslinked film comprising a polymeric binder on a substrate. The term “paint film” is synonymous with “coating film”.

The term, “effective amount” means the amount of any component required to achieve a desired result, e.g. in the case of a stabilizer composition, for the stabilizer composition to provide increased resistance to discoloration in the presence of UV-C (190-280 nm) light relative to a coating composition in the absence of the stabilizer composition. As described in the Examples section, changes in color (delta E or ΔE) or in Yellowness Index (delta YI or ΔYI) of the coating film after UV-C exposure are used to measure discoloration.

The term “hydrocarbyl” is a generic term encompassing aliphatic, alicyclic and aromatic groups having an all-carbon backbone and consisting of carbon and hydrogen atoms, except where otherwise stated. In certain cases, as defined herein, one or more of the carbon atoms making up the carbon backbone may be replaced by a specified atom or group of atoms. Examples of hydrocarbyl groups include alkyl, cycloalkyl, cycloalkenyl, carbocyclic aryl, alkenyl, alkynyl, alkylcycloalkyl, cycloalkylalkyl, cycloalkenylalkyl, and carbocyclic aralkyl, alkaryl, aralkenyl and aralkynyl groups. Such groups can be optionally substituted by one or more substituents as defined herein. Accordingly, the chemical groups or moieties discussed in the specification and claims should be understood to include the substituted or unsubstituted forms. The examples and preferences expressed below apply to each of the hydrocarbyl substituent groups or hydrocarbyl-containing substituent groups referred to in the various definitions of substituents for compounds of the formulas described herein unless the context indicates otherwise.

Preferred non-aromatic hydrocarbyl groups are saturated groups such as alkyl and cycloalkyl groups. Generally, and by way of example, the hydrocarbyl groups can have 12 to 60 carbon atoms, unless the context requires otherwise. Hydrocarbyl groups with from 12 to 30 carbon atoms are preferred.

Alkyl is intended to include linear, branched, or cyclic hydrocarbon structures and combinations thereof. Preferred alkyl groups are those of C₃₀ or below. Lower alkyl refers to alkyl groups of from 1 to 6 carbon atoms. Examples of lower alkyl groups include methyl, ethyl, propyl, isopropyl, butyl, sec-and tert-butyl, pentyl, hexyl and the like.

An aliphatic compound refers to a compound in which its main functional group is bonded to a saturated carbon atom. The rest of the carbon atoms can be aliphatic or aromatic. For example, benzyl alcohol is an aliphatic alcohol and benzyl amine is an aliphatic amine, because the hydroxy group and amino group are each bonded to saturated benzylic carbon atoms, respectively.

The term “interrupted by one or more heteroatoms” refers to an alkyl group containing one or more of —O—, —NH—, or —S— linking two carbon atoms in the alkyl group.

Alkoxy or alkoxyalkyl refers to groups of from 1 to 20 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like.

Acyl refers to formyl and to groups of 1, 2, 3, 4, 5, 6, 7, 8, 9, 10, 11 and 12 carbon atoms of a straight, branched, cyclic configuration, saturated, unsaturated and aromatic and combinations thereof, attached to the parent structure through a carbonyl functionality. Examples include acetyl, benzoyl, propionyl, isobutyryl, tert-butoxycarbonyl, benzyloxycarbonyl and the like. Lower-acyl refers to groups containing one to six carbons.

References to “carbocyclic” or “cycloalkyl” groups as used herein shall include both aromatic and non-aromatic ring systems, unless the context indicates otherwise. Thus, for example, the term includes within its scope aromatic, non-aromatic, unsaturated, partially saturated and fully saturated carbocyclic ring systems. In general, such groups may be monocyclic or bicyclic and may contain, for example, 3 to 12 ring members, more usually 5 to 10 ring members. Examples of monocyclic groups are groups containing 3, 4, 5, 6, 7, and 8 ring members, more usually 3 to 7, and preferably 5 or 6 ring members. Examples of bicyclic groups are those containing 8, 9, 10, 11 and 12 ring members, and more usually 9 or 10 ring members. Examples of non-aromatic carbocycle/cycloalkyl groups include c-propyl, c-butyl, c-pentyl, c-hexyl, and the like. Examples of C₇ to C₁₀ polycyclic hydrocarbons include ring systems such as norbornyl and adamantyl.

Aryl (carbocyclic aryl) refers to a 5- or 6-membered aromatic carbocycle ring containing; a bicyclic 9- or 10-membered aromatic ring system; or a tricyclic 13- or 14-membered aromatic ring system. The aromatic 6- to 14-membered carbocyclic rings include, e.g., substituted or unsubstituted phenyl groups, benzene, naphthalene, indane, tetralin, and fluorene.

Substituted hydrocarbyl, alkyl, aryl, cycloalkyl, alkoxy, etc. refer to the specific substituent wherein up to three H atoms in each residue are replaced with alkyl, halogen, haloalkyl, hydroxy, alkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, halobenzyl, heteroaryl, phenoxy, benzyloxy, heteroaryloxy, benzoyl, halobenzoyl, or lower alkylhydroxy.

The term “halogen” means fluorine, chlorine, bromine or iodine.

All numbers expressing quantities of ingredients, reaction conditions, etc. used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant figures and ordinary rounding approaches.

All ranges disclosed herein are inclusive of the endpoints, and the endpoints are independently combinable with each other (e.g., ranges of “less than or equal to 25 wt. %, or, more specifically, 5 wt. % to 20 wt. %”, is inclusive of the endpoints and all intermediate values of the ranges, including for example, “5 wt. % to 25 wt. %). Reference to “about” a value or parameter herein includes (and describes) embodiments that are directed to that value or parameter per se. The term “about” can also include the indicated amount ±10%, ±5%, or ±1%.

“At least one of” as used herein in connection with a list means that the list is inclusive of each element individually, as well as combinations of any two or more elements of the list, and combinations of at least one element of the list with any like elements not named.

“Combinations” is inclusive of blends, mixtures, reaction products, and the like. Singular articles indicate plural referents as well, unless the context clearly dictates otherwise. For example, the articles “a” and “an” and “the” as used herein do not denote a limitation of quantity and are to be construed to cover both the singular and the plural, unless otherwise indicated herein or clearly contradicted by context.

“Or” means “and/or” unless clearly stated otherwise, i.e. “A and/or B” means “A”, “B”, or “A and B”.

The coating compositions and methods disclosed herein may be embodied in many different forms and should not be construed as limited to the embodiments set forth herein. Instead, these embodiments are provided so that this disclosure will satisfy applicable legal requirements. Those skilled in the art will appreciate that while preferred embodiments are discussed in more detail below, multiple embodiments of the coating composition and methods described herein are contemplated. Thus, it should be noted that any element, step, component, or feature described with respect to one aspect or one embodiment of the disclosed coating compositions or methods can be combined with any other aspect or embodiment of the coating compositions or methods, unless otherwise stated. Additionally, any element, step, component, or feature recited in a list can be any one of the recited elements, steps, components, or features, or also be selected from a group including any two or more of the explicitly listed elements, components, or features, or may also be omitted from such list.

A coating composition comprising a polymeric binder and a stabilizer composition comprises at least one of a UV absorber, a hindered amine light stabilizer (HALS), or an inorganic UV blocker, wherein the coating composition is more resistant to discoloration when exposed to UV-C (190-280 nm) light compared to the coating composition in the absence of the stabilizer. As described in the Examples section, changes in color (delta E or ΔE) or in Yellowness Index (delta YI or ΔYI) of the coating film after UV-C exposure are used to measure discoloration. The stabilizer composition can provide resistance to other deleterious effects of UV-C light besides discoloration. For example, the stabilizer composition can provide resistance to cracking, crazing, gloss loss, hazing (reduced clarity and brilliance), and delamination of coating films from substrates, or from other coating film layers.

The stabilizer composition is present in an amount effective in providing resistance to discoloration in the presence of UV-C light relative to a coating composition in the absence of the stabilizer. The effective amount can be in the range of 0.005 to 10 wt. %, 0.01 to 5 wt. %, or 0.1 to 2 wt. %, based on the total solids contents of the stabilizer composition and the polymeric binder. Amounts of stabilizer in these ranges can provide increased resistance to discoloration in the presence of UV-C light relative to a coating composition in the absence of the stabilizer. The effective amount depends on many factors, for example the specific polymeric binder and other components of the coating composition, the application method, coating film thickness, and amount of UV-C exposure.

In any or all embodiments, the stabilizer composition can comprise a UV absorber. The UV absorber can be at least one of a 2-(2′-hydroxyphenyl)-s-triazine, 2-hydroxybenzophenone, 2-(2′-hydroxyphenyl)benzotriazole, or benzoxazinone. In the same or other embodiments, the stabilizer composition can comprise a mixture of UV absorbers. For example, in one embodiment, the stabilizer composition can comprise a 2-(2′-hydroxyphenyl)-s-triazine and a 2-(2′-hydroxyphenyl)benzotriazole.

In any or all embodiments, the UV absorber can be a 2-(2′-hydroxyphenyl)-s-triazine. 2-(2′-Hydroxyphenyl)-s-triazines are well known in the art. They are disclosed, for example, in U.S. Pat. Nos. 6,051,164 and 6,843,939, which are incorporated herein by reference. In any or all embodiments, the 2-(2′-hydroxyphenyl)-s-triazine can be a compound according to Formula (I):

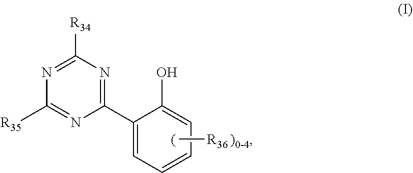

wherein each R₃₄ and R₃₅ is independently a C₆-C₁₀ aryl group, mono- or di-C₁-C₁₂ hydrocarbyl-substituted amino, C₂-C₁₂ alkanoyl, C₁-C₁₂ alkyl, C₁-C₁₀ acyl, or C₁-C₁₀ alkoxyl;

the C₆-C₁₀ aryl group is optionally substituted at from 1 to 3 substitutable positions with at least one of OH, halogen, C₁-C₁₂ alkyl, C₁-C₁₂ alkoxy, C₁₋₁₂ alkoxyester, C₂₋₁₂ alkanoyl, or phenyl, wherein the phenyl is optionally substituted at from 1 to 3 substitutable positions with at least one of OH, halogen, C₁₋₁₂ alkyl, C₁₋₁₂ alkoxy, C₁₋₁₂ alkoxyester, or C₂₋₁₂ alkanoyl; and each R36 is independently chosen from OH, halogen, C₁-C₁₂ alkyl, C₁-C₁₂ alkoxy, C₁-C₁₂ alkoxyester, C₂-C₁₂ alkanoyl, phenyl, or C₁-C₁₂ acyl.

The 2-(2′-hydroxyphenyl)-s-triazine can be, for example, at least one of: 4,6-bis-(2,4-dimethylphenyl)-2-(2-hydroxy-4-octyloxyphenyl)-s-triazine (CYASORB™ 1164); 4,6-bis-(2,4-dimethylphenyl)-2-(2,4-dihydroxyphenyl)-s-triazine; 2,4-bis(2,4-dihydroxyphenyl)-6-(4-chlorophenyl)-s-triazine; 2,4-bis[2-hydroxy-4-(2-hydroxy-ethoxy)phenyl]-6-(4-chlorophenyl)-s-triazine; 2,4-bis[2-hydroxy-4-(2-hydroxy-4-(2-hydroxy-ethoxy)phenyl]-6-(2,4-dimethylphenyl)-s-triazine; 2,4-bis[2-hydroxy-4-(2-hydroxyethoxy)phenyl]-6-(4-bromophenyl)-s-triazine; 2,4-bis[2-hydroxy-4-(2-acetoxyethoxy)phenyl]-6-(4-chlorophenyl)-s-triazine; 2,4-bis(2,4-dihydroxyphenyl)-6-(2,4-dimethylphenyl)-s-triazine; 2,4-bis(4-biphenylyl)-6-[2-hydroxy-4-[(octyloxycarbonyl)ethylideneoxy]phenyl]-s-triazine; 2,4-bis(4-biphenylyl)-6-[2-hydroxy-4-(2-ethylhexyloxy)phenyl]-s-triazine; 2-phenyl-4-[2-hydroxy-4-(3-sec-butyloxy-2-hydroxypropyloxy)phenyl]-6-[2-hydroxy-4-(3-sec-amyloxy-2-hydroxypropyloxy)phenyl]-s-triazine; 2,4-bis(2,4-dimethylphenyl)-6-[2-hydroxy-4-(3-benzyloxy-2-hydroxypropyloxy)phenyl]-s-triazine; 2,4-bis(2-hydroxy-4-n-butyloxyphenyl)-6-(2,4-di-n-butyloxyphenyl)-s-triazine; 2,4-bis(2,4-dimethylphenyl)-6-[2-hydroxy-4-(3-nonyloxy-2-hydroxypropyloxy)-5-α-cumylphenyl]-s-triazine; methylenebis-{2,4-bis(2,4-dimethylphenyl)-6-[2-hydroxy-4-(3-butyloxy-2-hydroxypropoxy)phenyl]-s-triazine}; methylene bridged dimer mixture bridged in the 3:5′, 5:5′ and 3:3′ positions in a 5:4:1 ratio; 2,4,6-tris(2-hydroxy-4-isooctyloxycarbonyliso-propylideneoxy-phenyl)-s-triazine; 2,4-bis(2,4-dimethylphenyl)-6-(2-hydroxy-4-hexyloxy-5-α-cumylphenyl)-s-triazine; 2-(2,4,6-trimethylphenyl)-4,6-bis[2-hydroxy-4-(3-butyloxy-2-hydroxypropyloxy)phenyl]-s-triazine; 2,4,6-tris[2-hydroxy-4-(3-sec-butyloxy-2-hydroxypropyloxy)-phenyl]-s-triazine; mixture of 4,6-bis-(2,4-dimethylphenyl)-2-(2-hydroxy-4-(3-dodecyloxy-2-hydroxypropoxy)phenyl)-s-triazine and 4,6-bis-(2,4-dimethylphenyl)-2-(2-hydroxy-4-(3-tridecyloxy-2-hydroxypropoxy)phenyl)-s-triazine (TINUVIN™ 400); 4,6-bis-(2,4-dimethylphenyl)-2-(2-hydroxy-4(3-(2-ethylhexyloxy)-2-hydroxypropoxy)-phenyl)-s-triazine; or 4,6-diphenyl-2-(4-hexyloxy-2-hydroxyphenyl)-s-triazine (TINUVIN™ 1577).

In any or all embodiments, the 2-(2′-hydroxyphenyl)-1,3,5-triazine can be at least one of:

4,6-diphenyl-2-(4-hexyloxy-2-hydroxyphenyl)-s-triazine (TINUVIN™ 1577),

4,6-bis-(2,4-dimethylphenyl)-2-(2-hydroxy-4-octyloxyphenyl)-s-triazine (CYASORB™ 1164),

2,4-bis[2-hydroxy-4-(2-hydroxy-4-(2-hydroxyethoxy)phenyl]-6-(2,4-dimethylphenyl)-s-triazine,

mixture of 4,6-bis(2,4-dimethylphenyl)-2-(2-hydroxy-4-(3-dodecyloxy-2-hydroxypropoxy)phenyl)-s-triazine and 4,6-bis-(2,4-dimethylphenyl)-2-(2-hydroxy-4-(3-tridecyloxy-2-hydroxypropoxy)phenyl)-s-triazine (TINUVIN™ 400),

4,6-bis(2,4-dimethylphenyl)-2-(2-hydroxy-4(3-(2-ethylhexyloxy)-2-hydroxypropoxy)-phenyl)-s-triazine (TINUVIN™ 405),

4,6-bis(2,4-dimethylphenyl)-2-(2-hydroxy-4(3-(2-ethylhexyloxy)-2-hydroxypropoxy)-phenyl)-s-triazine,

2,4-bis(2,4-dimethylphenyl)-6-[2-hydroxy-4-[(octyloxycarbonyl)ethylideneoxy]phenyl]-s-triazine (TINUVIN™ 479),

2,4-bis(4-biphenylyl)-6-[2-hydroxy-4-[(octyloxycarbonyl)ethylideneoxy]phenyl]-s-triazine,

2,4-bis(4-biphenylyl)-6-[2-hydroxy-4-(2-ethylhexyloxy)phenyl]-s-triazine (TINUVIN™ 1600),

2,4-bis(2-hydroxy-4-butyloxyphenyl)-6-(2,4-bis-butyloxyphenyl)-s-triazine (TRIAZINE™ 460),

2,4,6-tris[2-hydroxy-4-(3-sec-butyloxy-2-hydroxypropyloxy)-phenyl]-s-triazine, or

2,4,6-tris[2-hydroxy-4-[(octyloxycarbonyl)ethylideneoxy]phenyl]-s-triazine (TINUVIN™ 477).

In any or all embodiments, the UV absorber can be a 4-hydroxybenzophenone.

In any or all embodiments, the UV absorber can be a 2-hydroxybenzophenone. 2-Hydroxybenzophenones are well known in the art. They are disclosed, for example, in U.S. Pat. Nos. 2,976,259, 3,049,443, and 3,399,169, which are incorporated herein by reference. The 2-hydroxybenzophenone can be, for example, at least one of 2-hydroxy-4-methoxybenzophenone (CYASORB™ UV-9), 2,2′-dihydroxy-4-methoxybenzophenone (CYASORB™ UV-24), 2-hydroxy-4-octyloxybenzophenone (CYASORB™ UV-531), 2,2′-dihydroxy-4,4′-di-methoxybenzophenone, 2,2′-dihydroxybenzophenone, 2,2′,4,4′-tetrahydroxybenzophenone, 2,2′-dihydroxy-4,4′-dimethoxybenzophenone, 2,2′-dihydroxy-4,4′-diethoxybenzophenone, 2,2′-dihydroxy-4,4′-dipropoxybenzophenone, 2,2′-dihydroxy-4,4′-dibutoxybenzophenone, 2,2′-dihydroxy-4-methoxy-4′-ethoxybenzophenone, 2,2′-dihydroxy-4-methoxy-4′-propoxybenzophenone, 2,2′-dihydroxy-4-methoxy-4′-butoxybenzophenone, 2,2′-dihydroxy-4-ethoxy-4′-propoxybenzophenone, 2,2′-dihydroxy-4-ethoxy-4′-butoxybenzophenone, 2,3′-dihydroxy-4,4′-dimethoxybenzophenone, 2,3′-dihydroxy-4-methoxy-4′-butoxybenzophenone, 2-hydroxy-4,4′,5′-trimethoxybenzophenone, 2-hydroxy-4,4′,6′-tributoxybenzophenone, 2-hydroxy-4-butoxy-4′,5′-dimethoxybenzophenone, 2-hydroxy-4-ethoxy-2′,4′-dibutylbenzophenone, 2-hydroxy-4-propoxy-4′,6′-dichlorobenzophenone, 2-hydroxy-4-propoxy-4′,6′-dibromobenzophenone, 2,4-dihydroxybenzophenone, 2-hydroxy-4-ethoxybenzophenone, 2-hydroxy-4-propoxybenzophenone, 2-hydroxy-4-butoxybenzophenone, 2-hydroxy-4-methoxy-4′-methylbenzophenone, 2-hydroxy-4-methoxy-4′-ethylbenzophenone, 2-hydroxy-4-methoxy-4′-propylbenzophenone, 2-hydroxy-4-methoxy-4′-butylbenzophenone, 2-hydroxy-4-methoxy-4′-tert-butylbenzophenone, 2-hydroxy-4-methoxy-4′-chlorobenzophenone, 2-hydroxy-4-methoxy-2′-chlorobenzophenone, 2-hydroxy-4-methoxy-4′-bromobenzophenone, 2-hydroxy-4,4′-dimethoxybenzophenone, 2-hydroxy-4,4′-dimethoxy-3-methylbenzophenone, 2-hydroxy-4,4′-dimethoxy-2′-ethylbenzophenone, 2-hydroxy-4,4′,5′-trimethoxybenzophenone, 2-hydroxy-4-ethoxy-4′-methylbenzophenone, 2-hydroxy-4-ethoxy-4′-ethylbenzophenone, 2-hydroxy-4-ethoxy-4′-propylbenzophenone, 2-hydroxy-4-ethoxy-4′-butylbenzophenone, 2-hydroxy-4-ethoxy-4′-methoxybenzophenone, 2-hydroxy-4,4′-diethoxybenzophenone, 2-hydroxy-4-ethoxy-4′-propoxybenzophenone, 2-hydroxy-4-ethoxy-4′-butoxybenzophenone, 2-hydroxy-4-ethoxy-4′-chlorobenzophenone, or 2-hydroxy-4-ethoxy-4′-bromobenzophenone.

In any or all embodiments, the UV absorber can be a 2-2′-hydroxyphenyl)benzotriazole. The 2-hydroxyphenyl benzotriazole can be, for example, at least one of 2-(2′-hydroxy-5′-methylphenyl)benzotriazole (TINUVIN™ P), 2-(2′-hydroxy-5′-tert-butylphenyl)benzotriazole, 2-(2′-hydroxy-3′-methyl-5′-tert-butylphenyl)benzotriazole, 2-(2′-hydroxy-5′-cyclohexylphenyl)benzotriazole, 2-(2′-hydroxy-3′,5′-dimethylphenyl)benzotriazole, 2-(2′-hydroxy-5′-tert-butylphenyl)-5-chloro-benzotriazole, 2-(2′-hydroxy-5′-tert-octylphenyl)benzotriazole (CYASORB™ UV-5411), 2-(3′,5′-di-tert-butyl-2′-hydroxyphenyl)-5-chlorobenzotriazole, 2-(3′,5′-di-tert-amyl-2′-hydroxyphenyl)benzotriazole (CYASORB™ UV-2337), 2-(3′,5′-bis(α,α-dimethylbenzyl)-2′-hydroxyphenyl)benzotriazole (TINUVIN™ 900), 2-(3′-tert-butyl-2′-hydroxy-5′-(2-octyloxycarbonylethyl)phenyl)benzotriazole, 2,2′-methylenebis[4-(1,1,3,3-tetramethylbutyl)-6-benzotriazole-2-ylphenol], the transesterification product of 2-[3′-tert-butyl-5′-(2-methoxycarbonylethyl)-2′-hydroxyphenyl]-2H-benzotriazole with polyethylene glycol 300 (TINUVIN™ 1130), 2-[2′-hydroxy-3′-(α,α-dimethylbenzyl)-5′-(1,1,3,3-tetramethylbutyl)phenyl]benzotriazole, 5-trifluoromethyl-2-(2-hydroxy-3-α-cumyl-5-tert-octylphenyl)benzotriazole, 2-(2′-hydroxy-5′-(2-hydroxyethyl)phenyl)benzotriazole, 2-(2′-hydroxy-5′-(2-methacryloyloxyethyl)phenyl)benzotriazole, 2-(2′-hydroxy-3′-tert-butyl-5′-methylphenyl)-5-chlorobenzotriazole (TINUVIN™ 326), 2-(3′-sec-butyl-5′-tert-butyl-2′-hydroxyphenyl)benzotriazole, 2-(3′,5′-di-tert-butyl-2′-hydroxyphenyl)benzotriazole, 2-(3′-dodecyl-5′-methyl-2′-hydroxyphenyl)-benzotriazole, 2-(3′-tert-butyl-5′-(2-octyloxycarbonylethyl)-2′-hydroxyphenyl)-5-chlorobenzotriazole, 2-(5′-methyl-2′-hydroxyphenyl)benzotriazole, or 2-(5′-tert-butyl-2′-hydroxyphenyl)benzotriazole.

In any or all embodiments, the UV absorber can be a benzoxazinone. Benzoxazinones are well known in the art. They are disclosed, for example, in U.S. Pat. Nos. 4,446,262 and 6,774,232, which are incorporated herein by reference. The benzoxazinone can be, for example, at least one of 2-methyl-3,1-benzoxazin-4-one, 2-butyl-3,1-benzoxazin-4-one, 2-phenyl-3,1-benzoxazin-4-one, 2-(1- or 2-naphthyl)-3,1-benzoxazin-4-one, 2-(4-biphenyl)-3,1-benzoxazin-4-one, 2-p-nitrophenyl-3,1-benzoxazin-4-one, 2-m-nitrophenyl-3,1-benzoxazin-4-one, 2-p-benzoylphenyl-3,1-benzoxazin-4-one, 2-p-methoxyphenyl-3,1-benzoxazin-4-one, 2-O-methoxyphenyl-3,1-benzoxazin-4-one, 2-cyclohexyl-3,1-benzoxazin-4-one, 2p-(or m-)phthalimidephenyl-3,1-benzoxazin-4-one, N-phenyl-4-(3,1-benzoxazin-4-one-2-yl)phthalimide, N-benzoyl-4-(3,1-benzoxazine-4-one-2-yl)aniline, N-benzoyl-N-methyl-4-(3,1-benzoxazin-4-one-2-yl)-aniline, 2-p[p-(N-phenylcarbamonyl)phenyl]-3,1-benzoxazin-4-one, 2-[p-(N-phenyl N-methylcarbamoyl)phenyl]-3,1-benzoxazin-4-one, 2,2′-bis(3,1-benzoxazin-4-one), 2,2′-ethylenebis(3,1-benzoxazin-4-one), 2,2′-tetramethylenebis(3,1-benzoxazin-4-one), 2,2′-hexamethylenebis(3,1-benzoxazin-4-one), 2,2′-decamethylenebis(3,1-benzoxazin-4-one), 2,2′-p-phenylenebis(3,1-benzoxazin-4-one) (CYASORB™ UV-3638), 2,2′-m-phenylenebis(3,1-benzoxazin-4-one), 2,2′-(4,4′-diphenylene)bis(3,1-benzoxazin-4-one), 2,2′-(2,6- or 1,5-naphthalene)bis(3,1-benzoxazin-4-one), 2,2′-(2-methyl-p-phenylene)bis(3,1-benzoxazin-4-one), 2,2′-(2-nitro-p-phenylene)bis(3,1-benzoxazin-4-one), 2,2′-(2-chloro-p-phenylene)bis(3,1-benzoxazin-4-one), 2,2′-(1,4-cyclohexylene)bis(3,1-benzoxazin-4-one), N-p-(3,1-benzoxazin-4-on-2-yl)phenyl, 4-(3,1-benzoxazin-4-on-2-yl)phthalimide, N-p-(3,1-benzoxazin-4-on-2-yl)benzoyl, 4-(3,1-benzoxazin-4-on-2-yl)aniline, 1,3,5-tri(3,1-benzoxazin-4-on-2-yl)benzene. 1,3,5-tri(3,1-benzoxazin-4-on-2-yl)naphthalene, or 2,4,6-tri(3,1-benzoxazin-4-on-2-yl)naphthalene.

In any or all embodiments, the UV absorber is at least one of 2-hydroxy-4-octyloxybenzophenone (CYASORB™ UV-531), 4-hydroxybenzophenone, 2-(4,6-bis-(2,4-dimethylphenyl)-1,3,5-triazin-2-yl)-5-(isooctyloxy)phenol (CYASORB™ UV-1164L), mixture of 2-[4-[(2-hydroxy-3-dodecyloxypropyl)oxy]-2-hydroxyphenyl]-4,6-bis(2,4-dimethylphenyl)-1,3,5-triazine and 2-[4-[(2-hydroxy-3-tridecyloxypropyl)oxy]-2-hydroxyphenyl]-4,6-bis(2,4-dimethylphenyl)-1,3,5-triazine in 1-methoxy-2-propanol (TINUVIN™ 400), mixture of β-[3-(2-H-benzotriazole-2-yl)-4-hydroxy-5-tert-butylphenyl]-propionic acid poly(ethylene glycol) 300 ester and bis{β-[3-(2-H-benzotriazole-2-yl)-4-hydroxy-5-tert-butylphenyl]-propionic acid} poly(ethylene glycol) 300 ester (TINUVIN™ 1130).

In any or all embodiments, the stabilizer comprises an inorganic UV blocker. The inorganic UV blocker comprises at least one of titanium dioxide, zinc oxide, cerium(IV) oxide, or barium sulfate. An example of an inorganic UV blocker is cerium(IV) oxide, available from Solvay as RHODIGARD™ W185.

In any or all embodiments, the polymer composition can further comprise a hindered amine light stabilizer (HALS). The hindered amine light stabilizer can comprise at least one functional group according to Formula (II):

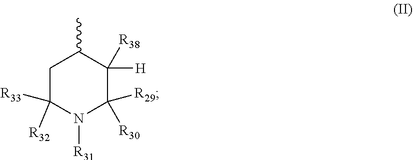

wherein:

R₃₁ is hydrogen, OH, C₁-C₂₀ hydrocarbyl, —CH₂CN, C₁-C₁₂ acyl, or C₁-C₁₈ alkoxy;

R₃₈ is hydrogen or C₁-C₈ hydrocarbyl; and

R₂₉, R₃₀, R₃₂, and R₃₃ are each independently C₁-C₂₀ hydrocarbyl, or R₂₉ and R₃₀ and/or R₃₂ and R₃₃ taken together with the carbon to which they are attached form a C₅-C₁₀ cycloalkyl; or

at least one functional group according to Formula (IIa):

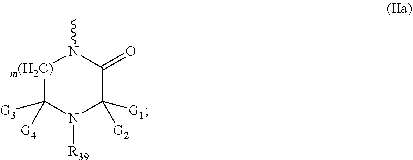

wherein:

m is an integer from 1 to 2;

R₃₉ is hydrogen, OH, C₁-C₂₀ hydrocarbyl, —CH₂CN, C₁-C₁₂ acyl, or C₁-C₁₈ alkoxy; and

G₁-G₄ are each independently C₁-C₂₀ hydrocarbyl.

The hindered amine light stabilizer (HALS) can be, for example, at least one of bis(2,2,6,6-tetramethylpiperidin-4-yl) sebacate (TINUVIN™ 770); bis(2,2,6,6-tetramethylpiperidin-4-yl)succinate; bis(1,2,2,6,6-pentamethylpiperidin-4-yl)sebacate; bis(1-octyloxy-2,2,6,6-tetramethylpiperidin-4-yl)sebacate (TINUVIN™ 123); bis(1,2,2,6,6-pentamethylpiperidin-4-yl) n-butyl 3,5-di-tert-butyl-4-hydroxybenzylmalonate; a condensate of 1-(2-hydroxyethyl)-2,2,6,6-tetramethyl-4-hydroxypiperidine and succinic acid; 2,2,6,6-tetramethylpiperidin-4-yl stearate; 2,2,6,6-tetramethylpiperidin-4-yl dodecanate; 1,2,2,6,6-pentamethylpiperidin-4-yl stearate; 1,2,2,6,6-pentamethylpiperidin-4-yl dodecanate; a condensate of N,N′-bis(2,2,6,6-tetramethylpiperidin-4-yl)hexamethylenediamine and 4-tert-octylamino-2,6-dichloro-1,3,5-triazine; tris(2,2,6,6-tetramethylpiperidin-4-yl) nitrilotriacetate; 4-benzoyl-2,2,6,6-tetramethylpiperidine; 4-stearyloxy-2,2,6,6-tetramethylpiperidine; bis(1,2,2,6,6-pentamethylpiperidyl)-2-n-butyl-2-(2-hydroxy-3,5-di-tert-butylbenzyl)malonate; 3-n-octyl-7,7,9,9-tetramethyl-1,3,8-triazaspiro [4.5]decan-2,4-dione; bis(1-octyloxy-2,2,6,6-tetramethylpiperidyl)sebacate; bis(1-octyloxy-2,2,6,6-tetramethylpiperidyl)succinate; a condensate of N,N′-bis(2,2,6,6-tetramethylpiperidin-4-yl)hexamethylenediamine and 4-morpholino-2,6-dichloro-1,3,5-triazine; a condensate of N,N′-bis(2,2,6,6-tetramethylpiperidin-4-yl)hexamethylenediamine and 4-morpholino-2,6-dichloro-1,3,5-triazine, methylated; a condensate of 2-chloro-4,6-bis(4-n-butylamino-2,2,6,6-tetramethylpiperidyl)-1,3,5-triazine and 1,2-bis(3-aminopropylamino)ethane; a condensate of 2-chloro-4,6-bis(4-n-butylamino-1,2,2,6,6-pentamethylpiperidyl)-1,3,5-triazine and 1,2-bis-(3-aminopropylamino)ethane; 8-acetyl-3-dodecyl-7,7,9,9-tetrmethyl-1,3,8-triazaspiro[4.5]decane-2,4-dione; 3-dodecyl-1-(2,2,6,6-tetramethylpiperidin-4-yl)pyrrolidin-2,5-dione; 3-dodecyl-1-(1-ethanoyl-2,2,6,6-tetramethylpiperidin-4-yl)pyrrolidin-2,5-dione; 3-dodecyl-1-(1,2,2,6,6-pentamethylpiperidin-4-yl)pyrrolidine-2,5-dione; a mixture of 4-hexadecyloxy- and 4-stearyloxy-2,2,6,6-tetramethylpiperidine, a mixture of 4-hexadecyloxy- and 4-stearyloxy-1,2,2,6,6-pentamethylpiperidine; a condensate of N,N′-bis(2,2,6,6-tetramethylpiperidin-4-yl)hexamethylenediamine and 4-cyclohexylamino-2,6-dichloro-1,3,5-triazine; a condensate of 1,2-bis(3-aminopropylamino)ethane, 2,4,6-trichloro-1,3,5-triazine and 4-butylamino-2,2,6,6-tetramethylpiperidine; 2-undecyl-7,7,9,9-tetramethyl-1-oxa-3,8-diaza-4-oxospiro[4.5]decane; oxo-piperanzinyl-triazines; a reaction product of 7,7,9,9-tetramethyl-2-cycloundecyl-1-oxa-3,8-diaza-4-oxospiro[4.5]decane and epichlorohydrin; 1,2,3,4-butanetetracarboxylic acid, 2,2,6,6-tetramethyl-4-piperidinyl tridecyl ester; 1,2,3,4-butanetetracarboxylic acid, 1,2,2,6,6-pentamethyl-4-piperidinyl tridecyl ester; tetrakis(2,2,6,6-tetramethylpiperidin-4-yl)-1,2,3,4-butanetetracarboxylate; tetrakis(1,2,2,6,6-pentamethylpiperidin-4-yl)-1,2,3,4-butanetetracarboxylate; 1,2,3,4-butanetetracarboxylic acid, polymer with β,β,β′,β′-tetramethyl-2,4,8,10-tetraoxaspiro[5.5]-undecane-3,9-diethanol, 2,2,6,6-tetramethylpiperdin-4-yl ester; 1,2,3,4-butanetetracarboxylic acid, polymer with β,β,β′,β′-tetramethyl-2,4,8,10-tetraoxaspiro[5.5]-undecane-3,9-diethanol, 1,2,2,6,6-pentamethylpiperdin-4-yl ester; bis(1-undecanoxy-2,2,6,6-tetramethylpiperidin-4-yl)carbonate; 1-(2-hydroxy-2-methylpropoxy)-2,2,6,6-tetramethyl-4-piperdinol; 1-(2-hydroxy-2-methylpropoxy)-4-octadecanoyloxy-2,2,6,6-tetramethylpiperidine; 1-(4-octadecanoyloxy-2,2,6,6-tetramethylpiperidin-1-yloxy)-2-octadecanoyloxy-2-methylpropane; 1-(2-hydroxyethyl)-2,2,6,6-tetramethyl-4-piperdinol; a reaction product of 1-(2-hydroxyethyl)-2,2,6,6-tetramethyl-4-piperdinol and dimethyl succinate; 2,2,4,4-tetramethyl-7-oxa-3,20-diazadispiro[5.1.11.2]heneicosan-21-one; esters of 2,2,6,6-tetramethyl-4-piperidinol with higher fatty acids; 3-dodecyl-1-(2,2,6,6-tetramethyl-4-piperidyl)pyrrolidine-2,5-dione; 1H-Pyrrole-2,5-dione, 1-octadecyl-, polymer with (1-methylethenyl)benzene and 1-(2,2,6,6-tetramethyl-4-piperidinyl)-1H-pyrrole-2,5-dione; 1,1′,1″-[1,3,5-triazine-2,4,6-triyl-tris[(cyclohexylimino)-2,1-ethanediyl]]tris[3,3,5,5- tetramethylpiperazin-2-one]; 1,1′,1″-[1,3,5-triazine-2,4,6-triyl-tris[(cyclohexylimino)-2,1-ethanediyl]]tris[3,3,4,5,5-pentamethylpiperazin-2-one]; the reaction product of 7,7,9,9-tetramethyl-2-cycloundecyl-1-oxa-3,8-diaza-4-oxospiro[4.5]decane and epichlorohydrin; the condensate of N,N′-bis(2,2,6,6-tetramethylpiperidin-4-yl)hexamethylenediamine and 4-cyclohexylamino-2,6-dichloro-1,3,5-triazine; the condensate of 1,2-bis(3-aminopropylamino)ethane, 2,4,6-trichloro-1,3,5-triazine and 4-butylamino-2,2,6,6-tetramethylpiperidine; the condensate of N,N′-bis(2,2,6,6-tetramethylpiperidin-4-yl)hexamethylenediamine and 4-morpholino-2,6-dichloro-1,3,5-triazine; the condensate of 2-chloro-4,6-bis(4-n-butylamino-2,2,6,6-tetramethylpiperidyl)-1,3,5-triazine and 1,2-bis(3-aminopropylamino)ethane; the condensate of 2-chloro-4,6-bis(4-n-butylamino-1,2,2,6,6-pentamethylpiperidyl)-1,3,5-triazine and 1,2-bis-(3-aminopropylamino)ethane; 2-[(2-hydroxyethyl)amino]-4,6-bis[N-(1-cyclohexyloxy-2,2,6,6-tetramethylpiperidin-4-yl)butylamino-1,3,5-triazine; propanedioic acid, [(4-methoxyphenyl)-methylene]-bis-(1,2,2,6,6-pentamethyl-4-piperidinyl) ester; benzenepropanoic acid, 3,5-bis(1,1-dimethylethyl)-4-hydroxy-, 1-[2-[3-[3,5-bis(1,1-dimethylethyl)-4-hydroxyphenyl]-1-oxopropoxy]ethyl]-2,2,6,6-tetramethyl-4-piperidinyl ester, N-(1-octyloxy-2,2,6,6-tetramethylpiperidin-4-yl)-N′-dodecyl-oxalamide; tris(2,2,6,6-tetramethylpiperidin-4-yl) nitrilotriacetate; 1,5-dioxaspiro{5,5}undecane-3,3-dicarboxylic acid, bis(1,2,2,6,6-pentamethyl-4-piperidinyl); 1,5-dioxaspiro{5,5}undecane-3,3-dicarboxylic acid, bis(2,2,6,6-tetramethyl-4-piperidinyl); the condensate of 1-(2-hydroxyethyl)-2,2,6,6-tetramethyl-4-hydroxypiperidine and succinic acid; the condensate of N,N′-bis(2,2,6,6-tetramethylpiperidin-4-yl)hexamethylenediamine and 4-tert-octylamino-2,6-dichloro-1,3,5-triazine; a mixture of 2,2,4,4-tetramethyl-21-oxo-7-oxa-3.20-diazaspiro(5.1.11.2)-heneicosane-20-propanoic acid-dodecyl ester and 2,2,4,4-tetramethyl-21-oxo-7-oxa-3.20-diazaspiro(5.1.11.2)-heneicosane-20-propanoic acid-tetradecyl ester; 1H,4H,5H,8H-2,3a,4a,6,7a,8a-hexaazacyclopenta[def]fluorene-4,8-dione, hexahydro-2,6-bis(2,2,6,6-tetramethyl-4-piperidinyl); polymethyl[propyl-3-oxy(2′,2′,6′,6′-tetramethyl-4,4′-piperidinyl)]siloxane; polymethyl[propyl-3-oxy(1′,2′,2′,6′,6′-pentamethyl-4,4′-piperidinyl)]siloxane; copolymer of methyl methacrylate with ethyl acrylate and 2,2,6,6-tetramethylpiperidin-4-yl acrylate; copolymer of mixed C₂₀ to C₂₄ alpha-olefins and (2,2,6,6-tetramethylpiperidin-4-yl)succinimide; 1,3-benzenedicarboxamide, N,N′-bis(2,2,6,6-tetramethyl-4-piperidinyl); 1,1′-1,10-dioxo-1,10-decanediyl)-bis(hexahydro-2,2,4,4,6-pentamethylpyrimidine; ethane diamide, N-(1-acetyl-2,2,6,6-tetramethylpiperidinyl)-N′-dodecyl; formamide, N,N′-1,6-hexanediylbis[N-(2,2,6,6-tetramethyl-4-piperidinyl) (UVINUL™ 4050); d-glucitol, 1,3:2,4-bis-O-(2,2,6,6-tetramethyl-4-piperidinylidene)-; 2,2,4,4-tetramethyl-7-oxa-3,20-diaza-21-oxo-dispiro[5.1.11.2]heneicosane; propanamide, 2-methyl-N-(2,2,6,6-tetramethyl-4-piperidinyl)-2-[(2,2,6,6-tetramethyl-4-piperidinyl)amino]-; 7-oxa-3,20-diazadispiro[5.1.11.2]heneicosane-20-propanoic acid, 2,2,4,4-tetramethyl-21-oxo-, dodecyl ester; N-(2,2,6,6-tetramethylpiperidin-4-yl)-β-aminopropionic acid dodecyl ester; N-(2,2,6,6-tetramethylpiperidin-4-yl)-N′-aminooxalamide; N-(2,2,6,6-tetramethyl-4-piperidinyl)-3-[(2,2,6,6-tetramethyl-4-piperidinyl)amino]-propanamide; 3-dodecyl-1-(1,2,2,6,6-pentamethylpiperidin-4-yl)pyrrolidine-2,5-dione, 3-dodecyl-1-(1-ethanoyl-2,2,6,6-pentamethylpiperidin-4-yl)pyrrolidine-2,5-dione; bis(2,2,6,6-tetramethylpiperidin-4-yl)succinate; bis(1,2,2,6,6-pentamethylpiperidin-4-yl) n-butyl 3,5-di-tert-butyl-4-hydroxybenzylmalonate; tris(2,2,6,6-tetramethylpiperidin-4-yl) nitrilotriacetate; 1,1′-(1,2-ethanediyl)bis(3,3,5,5-tetramethylpiperazin-2-one); 4-benzoyl-2,2,6,6-tetramethylpiperidine; 4-stearyloxy-2,2,6,6-tetramethylpiperidine; bis(1,2,2,6,6-pentamethylpiperidyl)-2-n-butyl-2-(2-hydroxy-3,5-di-tert-butylbenzyl)malonate; 3-n-octyl-7,7,9,9-tetramethyl-1,3,8-triazaspiro[4.5]decan-2,4-dione; bis(1-octyloxy-2,2,6,6-tetramethylpiperidyl)sebacate; bis(1-octyloxy-2,2,6,6-tetramethylpiperidyl)succinate; 8-acetyl-3-dodecyl-7,7,9,9-tetramethyl-1,3,8-triazaspiro[4.5]decane-2,4-dione; 3-dodecyl-1-(2,2,6,6-tetramethylpiperidin-4-yl)pyrrolidin-2,5-dione; 3-dodecyl-1-(1-ethanoyl-2,2,6,6-tetramethylpiperidin-4-yl)pyrrolidin-2,5-dione; 3-dodecyl-1-(1,2,2,6,6-pentamethylpiperidin-4-yl)pyrrolidine-2,5-dione; 2-undecyl-7,7,9,9-tetramethyl-1-oxa-3,8-diaza-4-oxospiro[4.5]decane; 1,5-dioxaspiro{5,5}undecane-3,3-dicarboxylic acid, bis(2,2,6,6-tetramethyl-4-piperidinyl) ester; 1,5-dioxaspiro{5,5}undecane-3,3-dicarboxylic acid, bis(1,2,2,6,6-pentamethyl-4-piperidinyl) ester; N¹-(β-hydroxyethyl)-3,3-pentamethylene-5,5-dimethylpiperazin-2-one; N¹-tert-octyl-3,3,5,5-tetramethyl-diazepin-2-one; N¹-tert-octyl-3,3-pentamethylene-5,5-hexamethylene-diazepin-2-one; N¹-tert-octyl-3,3-pentamethylene-5,5-dimethyl-piperazin-2-one; trans-1,2-cyclohexane-bis-(N¹-5,5-dimethyl-3,3-pentamethylene-piperazin-2-one); trans-1,2-cyclohexane-bis(N¹-3,3,5,5-dispiropentamethylene-piperazin-2-one); N¹-isopropyl-1,4-diazadispiro-3,3,5,5-pentamethylenepiperazin-2-one; N¹-isopropyl-1,4-diazadispiro-3,3-pentamethylene-5,5-tetramethylene-piperazin-2-one; N¹-isopropyl-5,5-dimethyl-3,3-pentamethylene-piperazin-2-one; trans-1,2-cyclohexane-bis-N¹-(dimethyl-3,3-pentamethylene-piperazin-2-one); N¹-octyl-5,5-dimethyl-3,3-pentamethylene-1,4-diazepin-2-one; N¹-octyl -1,4-diazadispiro-(3,3,5,5)pentamethylene-1,5-diazepin-2-one; a condensate of N,N′-bis(2,2,6,6-tetramethyl-1-(propyloxy)-piperidin-4-yl)hexamethylenediamine, N-butyl-1-propyloxy-2,2,6,6-tetramethyl-4-piperidinamine, di-n-butyl amine, and 2,4,6-trichloro-1,3,5-triazine (TINUVIN™ NOR HALS 371); N,N′-bis(2,2,6,6-tetramethyl-4-piperidin-4-yl)hexamethylene diamine, polymer with 2,4,6-trichloro-1,3,5-triazine, reaction products with 3-bromo-l-propene, di-n-butylamine, and N-butyl-2,2,6,6-tetramethyl-4-piperidinamine, oxidized, hydrogenated (TINUVIN™ XT 200); TINUVIN™ XT-850/XT-855; or N-butyl-2,2,6,6-tetramethyl-4-piperidinamine-2,4,6-trichloro-1,3,5-triazine (FLAMESTAB™ NOR 116).

In any or all embodiments, the hindered amine light stabilizer (HALS), can be at least one of:

bis(2,2,6,6-tetramethylpiperidin-4-yl) sebacate (TINUVIN™ 770);

bis(2,2,6,6-tetramethylpiperidin-4-yl) succinate;

bis(1,2,2,6,6-pentamethylpiperidin-4-yl) sebacate;

bis(1-octyloxy-2,2,6,6-tetramethylpiperidyl) succinate;

bis(1-octyloxy-2,2,6,6-tetramethylpiperidin-4-yl) sebacate (TINUVIN™ 123);

bis(1,2,2,6,6-pentamethylpiperidin-4-yl) n-butyl 3,5-di-tert-butyl-4-hydroxybenzylmalonate;

a condensate of 1-(2-hydroxyethyl)-2,2,6,6-tetramethyl-4-hydroxypiperidine and succinic acid (TINUVIN™ 622);

2,2,6,6-tetramethylpiperidin-4-yl stearate;

2,2,6,6-tetramethylpiperidin-4-yl dodecanate;

1,2,2,6,6-pentamethylpiperidin-4-yl stearate;

1,2,2,6,6-pentamethylpiperidin-4-yl dodecanate;

a condensate of N,N′-bis(2,2,6,6-tetramethylpiperidin-4-yl)hexamethylenediamine and 4-tert-octylamino-2,6-dichloro-1,3,5-triazine (CHIMASSORB™ 944);

tris(2,2,6,6-tetramethylpiperidin-4-yl) nitrilotriacetate;

4-stearyloxy-2,2,6,6-tetramethylpiperidine;

a condensate of N,N′-bis(2,2,6,6-tetramethylpiperidin-4-yl)hexamethylenediamine and 4-morpholino-2,6-dichloro-1,3,5-triazine (CYASORB™ UV-3346);

a condensate of N,N′-bis(2,2,6,6-tetramethylpiperidin-4-yl)hexamethylenediamine and 4-morpholino-2,6-dichloro-1,3,5-triazine, methylated (CYASORB™ UV-3529);

a condensate of 2-chloro-4,6-bis(4-n-butylamino-2,2,6,6-tetramethylpiperidyl)-1,3,5-triazine and 1,2-bis(3-aminopropylamino)ethane (CHIMASSORB™ 119);

a condensate of 2-chloro-4,6-bis(4-n-butylamino-1,2,2,6,6-pentamethylpiperidyl)-1,3,5-triazine and 1,2-bis-(3-aminopropylamino)ethane;

a condensate of N,N′-bis(2,2,6,6-tetramethylpiperidin-4-yl)hexamethylenediamine, N-butyl-2,2,6,6-tetramethyl-4-piperidinamine, di-n-butyl amine, and 2,4,6-trichloro-1,3,5-triazine (CHIMASSORB™ 2020);

a mixture of 4-hexadecyloxy- and 4-stearyloxy-2,2,6,6-tetramethylpiperidine (CYASORB™ UV-3853);

a mixture of 4-hexadecyloxy- and 4-stearyloxy-1,2,2,6,6-pentamethylpiperidine;

a condensate of N,N′-bis(2,2,6,6-tetramethylpiperidin-4-yl)hexamethylenediamine and 4-cyclohexylamino-2,6-dichloro-1,3,5-triazine;

a condensate of 1,2-bis(3-aminopropylamino)ethane, 2,4,6-trichloro-1,3,5-triazine, and 4-butylamino-2,2,6,6-tetramethylpiperidine;

a condensate of N,N′-bis(2,2,6,6-tetramethylpiperidin-4-yl)hexamethylenediamine and 4-cyclohexylamino-2,6-dichloro-1,3,5-triazine;

tetrakis(2,2,6,6-tetramethylpiperidin-4-yl)-1,2,3,4-butanetetracarboxylate;

tetrakis(1,2,2,6,6-pentamethylpiperidin-4-yl)-1,2,3,4-butanetetracarboxylate;

1,2,3,4-butanetetracarboxylic acid, 2,2,6,6-tetramethylpiperidinyl-4-yl tridecyl ester;

1,2,3,4-butanetetracarboxylic acid, 1,2,2,6,6-pentamethylpiperidin-4-yl tridecyl ester;

formamide, N,N′-1,6-hexanediylbis[N-(2,2,6,6-tetramethylpiperidin-4-yl) (UVINUL™ 4050);

a condensate of N,N′-bis(2,2,6,6-tetramethyl-1-(propyloxy)-piperidin-4-yl)hexamethylenediamine, N-butyl-1-propyloxy-2,2,6,6-tetramethyl-4-piperidinamine, di-n-butyl amine, and 2,4,6-trichloro-1,3,5-triazine (TINUVIN™ NOR HALS 371;

N,N′-bis(2,2,6,6-tetramethyl-4-piperidin-4-yl)hexamethylene diamine, polymer with 2,4,6-trichloro-1,3,5-triazine, reaction products with 3-bromo-l-propene, di-n-butylamine, and N-butyl-2,2,6,6-tetramethyl-4-piperidinamine, oxidized, hydrogenated (TINUVIN™ XT 200);

TINUVIN™ XT-850/XT-855); or

N¹,N¹′-1,2-ethanediylbis(1,3-propanediamine), reaction products with cyclohexane and peroxidized N-butyl-2,2,6,6-tetramethyl-4-piperidinamine-2,4,6-trichloro-1,3,5-triazine (FLAMESTAB™ NOR 116).

In any or all embodiments, the hindered amine light stabilizer (HALS) can be at least one of a mixture of 2,2,6,6-tetramethylpiperidin-4-yl esters of C₁₂-C₂₁ saturated and C₁₈ unsaturated fatty acids (CYASORB™ UV-3853), a mixture of bis(1,2,2,6,6-pentamethylpiperidin-4-yl) sebacate and methyl 1,2,2,6,6-pentamethylpiperidin-4-yl sebacate (TINUVIN™ 292), or bis(1-octyloxy-2,2,6,6-tetramethylpiperidin-4-yl) sebacate (TINUVIN™ 123).

Combinations of stabilizers can be used. In any or all embodiments, the stabilizer composition can comprise a mixture of UV absorbers, for example a 2-2′-hydroxyphenyl)-s-triazine and a 2-(2′-hydroxyphenyl)benzotriazole. In any or all embodiments, the stabilizer composition can also comprise a UV absorber and a hindered amine light stabilizer (HALS). In any or all embodiments, the stabilizer composition can also comprise an inorganic UV blocker in combination with a UV absorber, a hindered amine light stabilizer (HALS), or mixture thereof.

The coating composition comprises a polymeric binder. The polymeric binder serves as a solid matrix for additives, such as pigments. There is no limit on what the polymeric binder can be. The polymeric binder can be a natural polymer, a semi-synthetic polymer derived from a natural polymer, or a synthetic polymer. The polymeric binder can be a thermoplastic or a thermoset, or crosslinkable. Some examples (non-exhaustive) of thermoplastics for use in any embodiment are provided below:

1. Polymers of monoolefins and diolefins, for example polypropylene (PP), polyisobutylene, polybut-1-ene, poly-4-methylpent-1-ene, polyvinylcyclohexane, polyisoprene or polybutadiene, as well as polymers of cycloolefins, e.g. of cyclopentene or norbornene, polyethylene (optionally crosslinked), e.g. high density polyethylene (HDPE), high density and high molecular weight polyethylene (HDPE-HMW), high density and ultrahigh molecular weight polyethylene (HDPE-UHMW), medium density polyethylene (MDPE), low density polyethylene (LDPE), linear low density polyethylene (LLDPE), (VLDPE) and (ULDPE).

Polyolefins, i.e. the polymers of monoolefins exemplified in the preceding paragraph, especially polyethylene and polypropylene, can be prepared by different, and especially by the following, methods:

a) radical polymerisation (often under high pressure and at elevated temperature),

b) catalytic polymerisation using a catalyst that normally contains one or more than one metal of Groups IVb, Vb, VIb, or VIII of the Periodic Table. These metals usually have one or more than one ligand, typically oxides, halides, alcoholates, esters, ethers, amines, alkyls, alkenyls and/or aryls that may be either π or σ-coordinated. These metal complexes may be in the free form or fixed on substrates, such as activated magnesium chloride, titanium(III) chloride, alumina or silicon oxide. These catalysts can be soluble or insoluble in the polymerisation medium. The catalysts can be used alone or in combination with activators, e.g. metal alkyls, metal hydrides, metal alkyl halides, metal alkyl oxides or metal alkyloxanes, said metals being elements of Groups Ia, IIa, and/or IIIa of the Periodic Table. The activators can be modified with further ester, ether, amine or silyl ether groups. These catalyst systems are known as Phillips, Standard Oil Indiana, Ziegler(-Natta), TNZ (Dupont), metallocene or single site catalysts (SSC).

2. Mixtures of the polymers mentioned under 1), e.g. mixtures of polypropylene and polyisobutylene, polypropylene and polyethylene (e.g. PP/HDPE, PP/LDPE) and mixtures of different types of polyethylene (e.g. LDPE/HDPE).

3. Copolymers of monoolefins and diolefins with each other or with other vinyl monomers, for example ethylene/propylene copolymers linear low density polyethylene (LLDPE) and mixtures thereof with low density polyethylene (LDPE), propylene/but-1-ene copolymers, propylene/isobutylene copolymers, ethylene/but-1-ene copolymers, ethylene/hexene copolymers, ethylene/methylpentene copolymers, ethylene/heptene copolymers, ethylene/octene copolymers, ethylene/vinylcyclohexane copolymers. ethylene/cycloolefin copolymers (COC, e.g. ethylene/norbornene), ethylene/α-olefin copolymers, where the /α-olefin is generated in-situ, propylene/butadiene copolymers, isobutylene/isoprene copolymers, ethylene/vinylcyclohexene copolymers, ethylene/alkyl acrylate copolymers, ethylene/alkyl methacrylate copolymers, ethylene/vinyl acetate copolymers, or ethylene/acrylic acid copolymers and their salts (ionomers) as well as terpolymers of ethylene with propylene and a diene such as hexadiene, dicyclopentadiene, or ethylidene-norbornene, and mixtures of these copolymers with each other and with polymers mentioned in 1) above, e.g. polypropylene/ethylene-propylene copolymers, LDPE/ethylene-vinyl acetate copolymers (EVA), LDPE/ethylene-acrylic acid copolymers (EAA), LLDPE/EVA, LLDPE/EAA, and alternating or random polyalkylene/carbon monoxide copolymers and mixtures with other polymers, e.g. polyamides.

4. Hydrocarbon resins (for example C₅-C₉) including hydrogenated modifications thereof (e.g. tackifiers) and mixtures of polyalkylenes and starch. Homopolymers, copolymers, and stereoblock copolymers from any of Items 1-4 above can have any stereostructure including syndiotactic, isotactic, hemisotactic, or atactic, especially atactic.

5. Polystyrene, poly(p-methyl styrene), poly(α-methyl styrene).

6. Aromatic homopolymers, copolymers, and stereoblock copolymers derived from vinyl aromatic monomers including styrene, α-methylstyrene, isomers of vinyl toluene, especially p-vinyltoluene, isomers of ethyl styrene, propyl styrene, vinyl biphenyl, vinyl naphthalene, vinyl anthracene, and mixtures thereof. The homopolymers and copolymers can have any stereostructure including syndiotactic, isotactic, hemisotactic, or atactic, especially atactic.

7. Copolymers including aforementioned vinyl aromatic monomers and comonomers selected from ethylene, propylene, dienes, (meth)acrylonitriles, (meth)acrylic acids, maleic anhydrides, maleimides, vinyl acetate, vinyl chloride, acrylic derivatives, and mixtures thereof, e.g. styrene/butadiene, styrene/acrylonitrile, styrene/ethylene (interpolymers), acrylonitrile/ethylene/styrene (AES), styrene/alkyl methacrylate, styrene/butadiene/alkyl acrylate, styrene/butadiene/alkyl meth acrylate styrene/maleic anhydride, styrene/acrylonitrile/methyl acrylate; mixtures of high impact strength of styrene copolymers and other polymers, e.g. a polyacrylate, a diene polymer or an ethylene/propylene/diene terpolymer; and block copolymers of styrene, e.g. styrene/butadiene/styrene, styrene/isoprene/styrene, styrene/ethylene/butylene/styrene, and styrene/ethylene/propylene/styrene.

8. Hydrogenated aromatic polymers derived from hydrogenation of the polymers of Item 6 above, especially polycyclohexylethylene (PCHE) prepared by hydrogenation of atactic polystyrene, also referred to as polyvinylcyclohexane (PVCH).

9. Hydrogenated aromatic polymers derived from hydrogenation of the copolymers of Item 6a above. The copolymers can be stereoblock copolymers, and can have any stereostructure including syndiotactic, isotactic, hemisotactic, or atactic, especially atactic.

10. Graft copolymers of vinyl aromatic monomers such as styrene or α-methylstyrene (high impact polystyrene, HIPS), e.g. styrene on polybutadiene, styrene on polybutadiene-styrene or polybutadiene-acrylonitrile copolymers; styrene and acrylonitrile (or methacrylonitrile) on polybutadiene (ABS): styrene, acrylonitrile, and methyl methacrylate on polybutadiene (MBS), styrene and maleic anhydride on polybutadiene, styrene, acrylonitrile and maleic anhydride or maleimide on polybutadiene, styrene and maleimide on polybutadiene, styrene and alkyl acrylates or methacrylates on polybutadiene, styrene and acrylonitrile on ethylene/propylene/diene terpolymers; styrene and acrylonitrile on polyalkyl acrylates or polyalkyl methacrylates, e.g. acrylonitrile and styrene on butyl acrylate (ASA), styrene and acrylonitrile on acrylate/butadiene copolymers, and mixtures with the copolymers listed under Item 6 above, e.g. the copolymer mixtures ABS, MBS, ASA, and AES.

11. Halogen-containing polymers such as polychloroprene, chlorinated rubbers, chlorinated and brominated copolymers of isobutylene-isoprene (halobutyl rubber), chlorinated (CPE) or sulfo-chlorinated polyethylene, copolymers of ethylene and chlorinated ethylene, epichlorohydrin homo- and co-polymers, especially polymers of halogen-containing vinyl monomers, e.g. polyvinyl chloride (PVC), polyvinylidene chloride (PVDC), polyvinyl fluoride (PVF), polyvinylidene fluoride (PVDF), and copolymers thereof such as vinyl chloride/vinylidene chloride, vinyl chloride/vinyl acetate, or vinylidene chloride/vinyl acetate copolymers.

12. Polymers of α,β-unsaturated acids and derivatives thereof, e.g. polyacrylates and polymethacrylates, polymethyl methacrylates, polyacrylamides, and polyacrylonitriles, optionally impact-modified with butyl acrylate, carboxylated styrene/butadiene copolymers and emulsions thereof, siloxane-modified polyacrylates, and fluorine-modified polyacrylates.

13. Copolymers of the monomers under Item 9 with each other or with other unsaturated monomers, e.g. acrylonitrile/butadiene copolymers, acrylonitrile/alkyl acrylate copolymers, acrylonitrile/alkoxyalkyl acrylate copolymers, or acrylonitrile/alkyl methacrylate/butadiene terpolymers.

14. Polymers derived from unsaturated alcohols and amines, or acyl derivatives or acetals thereof, e.g. polyvinyl alcohol (PVA), polyvinyl acetate (PVA), polyvinyl stearate, polyvinyl benzoate, polyvinyl maleate. polyvinyl butyral, polyallyl phthalate, or polyallyl melamine, and copolymers with the monomers under Item 1 above.

15. Homopolymers and copolymers of epoxides such as polyalkylene glycols, polyethylene oxide, polypropylene oxide, or copolymers thereof with bisglycidyl ethers.

16. Polyacetals such as polyoxymethylene (POM), polyoxymethylene copolymers with ethylene oxide, and polyacetals modified with thermoplastic polyurethanes, acrylates, or MBS.

17. Polyphenylene oxides (PPO), polyphenylene sulfides (PPS), and mixtures of polyphenylene oxides with styrene polymers or polyamides.

18. Polyurethanes (PUR) derived from hydroxyl-terminated polyethers, polyesters, or polybutadienes as the nucleophilic comonomer and aliphatic or aromatic polyisocyanates as the electrophilic comonomer, and oligomeric precursors thereof. The polyisocyanate can be, for example, hexamethylene diisocyanate, octamethylene diisocyanate, decamethylene diisocyanate, dodecamethylene diisocyanate, tetradecamethylene diisocyanate, trimethylhexane diisocyanate, tetramethylhexane diisocyanate, isophorone diisocyanate, 2-isocyanatopropylcyclohexyl isocyanate, dicyclohexylmethane-2,4′-diisocyanate, dicyclohexylmethane-4,4′-diisocyanate, 1,4- or 1,3-bis(isocyanatomethyl)cyclohexane, 1,4- or 1,3- or 1,2-diisocyanatocyclohexane, 2,4- or 2,6-diisocyanato-1-methylcyclo-hexane, diisocyanates derived from dimer fatty acids, as sold under the trade designation DDI 1410 by Henkel, 1,8-diisocyanato-4-isocyanato-methyloctane, 1,7-diisocyanato-4-isocyanatomethylheptane,1-isocyanato-2-(3-isocyanatopropyl)cyclohexane, tetramethylxylylene diisocyanates (TMXDI™), toluene diisocyanate (TDI), methylene diphenyl diisocyanate (MDI), isocyanurate trimers thereof, and mixtures thereof. The polyurethane can also be an acrylated polyurethane with pendant and/or terminal double bonds.

19. Polyamides (PA) and copolyamides derived from diamines, dicarboxylic acids and/or aminocarboxylic acids or the corresponding lactams, e.g. polyamides 4, 6, 6/6, 6/10, 6/9, 6/12, 4/6, 12/12, 11, or 12, aromatic polyamides prepared from m-xylene diamine and adipic acid, polyamides prepared from hexamethylene diamine and isophthalic and/or terephthalic acid, optionally with an elastomer modifier, e.g. poly-2,4,4-trimethylhexamethylene terephthalamide or poly-m-phenylene isophthalamide, block copolymers of the aforementioned polyamides with polyolefins, olefin copolymers, ionomers, or chemically bonded or grafted elastomers, or with polyethers, e.g. with polyethylene glycol, polypropylene glycol or polytetramethylene glycol as well as polyamides or copolyamides modified with EPDM or ABS, and polyamides condensed during processing (RIM polyamides).

20. Polyureas, polyimides, polyamide-imides, polyetherimides, polyesterimides, polyhydantoins, and polybenzimidazoles.

21. Polyesters prepared from dicarboxylic acids and diols and/or from hydroxycarboxylic acids or the corresponding lactones, e.g. polyethylene terephthalate (PET), polybutylene terephthalate (PBT), poly-1,4-dimethyolcyclohexane terephthalate, polyalkylene naphthalate (PAN) and polyhydroxybcnzoates, block copolyether esters prepared from hydroxyl-terminated polyethers, and polyesters modified with polycarbonates or MBS. Copolyesters can comprise, e.g. polybutylene succinate/terephthalate, polybutylene adipate/terephthalate, polytetramethylene adipate/terephthalate, polybutylene succinate/adipate, polybutylene succinate/carbonate, poly-3-hydroxybutyrate/octanoate copolymer, poly-3-hydroxybutyrate/hexanoate/decanoate terpolymer. The polyester can also be a poly(hydroxyalkanoate), e.g. poly(propiolactone), poly(butyrolactone), poly(pivalolactone), poly(valerolactone), and poly(caprolactone), polyethylene succinate, polypropylene succinate, polybutylene succinate, polyhexamethylene succinate, polyethylene adipate, polypropylene adipate, polybutylene adipate, polyethylene oxalate, polypropylene oxalate, polybutylene oxalate, polyhexamethylene oxalate, polyethylene sebacate, polypropylene sebacate, polybutylene sebacate, polylactic acid (PLA), optionally modified with polycarbonate or MBS. The term “polylactic acid (PLA)” refers to poly(L-lactide), optionally blended with other polymers, a copolymer of lactic acid or lactide with other monomers, such as hydroxyl-carboxylic acids, e.g. glycolic acid, 3-hydroxybutyric acid, 4-hydroxybutyric acid, 4-hydroxyvaleric acid, 5-hydroxyvaleric acid, 6-hydroxyvaleric acid, and cycle forms thereof. The terms “lactic acid” and “lactide” refer collectively to L-lactic acid, D-lactic acid, and mixtures thereof, e.g. L-lactide, D-lactide, meso-lactide, and mixtures thereof.

22. Polycarbonates (PC) and polyester carbonates.

23. Polyketones and polyether ketones.

24. Polysulfones and polyether sulfones.

25. Natural polymers, such as cellulose, latex rubber, gelatin, and chemically modified homologous derivatives thereof, e.g. cellulose acetates, cellulose propionates, cellulose butyrates, cellulose ethers such as methyl cellulose, rosins, and natural oils such as linseed oil, castor oil, and tung oil.

26. Natural and synthetic organic materials which are non-polymeric, e.g. mineral oils, animal and vegetable fats, oils, and waxes, and oils and waxes based on synthetic esters, e.g. phthalate, adipate, phosphates, and trimellitate esters, and mixtures of these esters with mineral oils.

27. Blends of any combinations of the aforementioned polymers, e.g. PP/EPDM, polyamide/EPDM or ABS, PVC/EVA, PVC/ABS, PVC/MBS, PC/ABS, PBT/ASA, PC/PBT, PVC/CPE, PVC/acrylates, POM/thermoplastic PUR, POM/acrylate, POM/MBS, PPO/HIPS, PPO/PA 6/6 and copolymers, PA/HDPE, PA/PP, PA/PPO, PBT/PC/ABS or PBT/PET/PC.

In any or all embodiments, the polymeric binder comprises a polyacrylate, a polyurethane, or mixture thereof.

The polymeric binder can also be a thermoset. The thermoset polymeric binder can be self-crosslinking or can be combined with a crosslinker. Some examples (non-exhaustive) of thermoset coating compositions suitable for use in any embodiment are provided below:

1. Crosslinked polymers derived from aldehydes, especially formaldehyde I combination with phenols, ureas, melamines, e.g. phenol/formaldehyde resins, urea/formaldehyde resins, melamine/formaldehyde resins, and alkoxylated (etherified) derivatives thereof.

2. Drying and non-drying alkyd resins.

3. Unsaturated polyester resins derived from copolyesters of saturated and unsaturated dicarboxylic acids with polyhydric alcohols and vinyl compounds as crosslinking agents, and low flammability halogen-containing modifications thereof.

4. Crosslinkable acrylic resins derived from substituted acrylates, e.g. epoxy acrylates, urethane acrylates, and polyester acrylates.

5. Alkyd resins, polyester resins, and acrylate resins crosslinked with melamine resins, urea resins, epoxy resins, or polyisocyanates.

6. Crosslinked epoxy resins derived from aliphatic, cycloaliphatic, heterocyclic, or aromatic glycidyl compounds, e.g. products of diglycidyl ethers of bisphenol A or bisphenol F, crosslinked with hardeners such as anhydrides or polyamines, optionally with accelerators.

Other examples of thermosets suitable for use in any embodiment include the following coating compositions.

1. Ambient or high temperature crosslinkable alkyd, acrylate, polyester, epoxy, or melamine resins, or mixtures thereof, optionally with curing catalyst.

2. Two-component (2K) polyurethane coatings comprising as one component, acrylate, polyester, or polyether polyols, and as the second component aliphatic or aromatic isocyanates, isocyanurates or polyisocyanates.

3. One-component (1K) polyurethane coatings comprising blocked isocyanates, isocyanurates or polyisocyanates, which deblocked during baking, optionally in combination with a melamine resins.

4. One-component (1K) polyurethane coatings comprising combinations of a trisalkoxycarbonyltriazine (TACT) crosslinker and an acrylate, polyester, or polyether polyol, or mixtures thereof.

5. One-component (1K) polyurethane coatings comprising aliphatic or aromatic urethane acrylates or polyurethane acrylates having free amino groups in the urethane and melamine resins or polyether resins, optionally with a curing catalyst.

6. Two-component (2K) coatings comprising polyketimines in combination with aliphatic or aromatic isocyanates, isocyanurates, or polyisocyanates.

7. Two-component (2K) coatings comprising polyketimines and an unsaturated acrylate resin, polyacetoacetate resin, or methacrylamidoglycolate methyl ester.

8. Two-component (2K) coatings comprising carboxyl- or amino-functional polyacrylates and polyepoxides.

9. Two-component (2K) coatings comprising anhydride-functional polyacrylates and polyol or polyamine.

10. Two-component (2K) coatings comprising anhydride-functional polyacrylates and polyepoxides.

11. Two-component (2K) coatings comprising polyoxazolines and anhydride-functional acrylate resins, unsaturated polyacrylates, or aliphatic or aromatic isocyanates, isocyanurates or polyisocyanates.

12. Two-component (2K) coatings comprising unsaturated polyacrylates and polymalonates.

13. Two-component (2K) coatings comprising polyacrylate polyols in combination with etherified melamine resins.

The coating composition can be cured by evaporation of a liquid medium, by chemical crosslinking or by a combination of evaporation of a liquid medium and chemical crosslinking to form a coating film. It can be cured at ambient temperature or by heating for a time and temperature necessary to remove the liquid medium and/or chemically crosslink the coating. In radiation cured (rad cure), crosslinking is produced by actinic radiation, e.g. by UV light (UV-cured coating), in the presence of a photoinitiator.

Coating films serve to protect substrates from the environment and to provide an aesthetic effect. They can be classified according to their end-uses. The end-use application can be, for example, transportation coatings, automotive coatings, refinish coatings, general industrial coatings, coil coatings, heavy-duty maintenance coatings, marine coatings, architectural coatings, wood stains and varnishes, paper coatings, or inks. The coating compositions can also be for recording media, e.g. reprographic paper, ink-jet paper, photographic paper, pressure-sensitive paper, heat-sensitive paper, and microcapsule paper. A coating film can also be a component of a multi-layer coating, and be disposed between a substrate and an outer coating film, or between two other coating films. As defined herein, the coating film can also fill a space between two substrates, or two surfaces of a substrate. Thus, the coating film can be part of a laminate. The end-use of the coating composition can also be as adhesives, sealants, liquid gaskets, or knifing fillers.

The coating compositions can also be classified according to its physical form, method of curing, its function, or some other characteristic. For example, the coating can utilize a liquid medium as a diluent (solvent-borne coating), can utilize an aqueous liquid medium (water-borne coating), can be a suspension in a liquid plasticizer (e.g. a PVC plastisol), or can be in the form of a particulate solid (powder coating). The polymeric binder can be at least partially dissolved in the liquid medium, or alternatively, dispersed in the liquid medium. The coating composition can also be a neat thermoplastic melt that is co-extruded onto a substrate layer and hardens upon cooling, i.e. a coextruded film.

The coating can be an anti-corrosion coating for metal substrates, for example a primer or electrocoat coating (e-coating). Electrocoats are water-borne coatings in which the polymeric binder adheres to the substrate by means of an electric current. The electrocoat can be anodically deposited or cathodically deposited. The coating can be a primer surfacer for application to a primer or electrocoat coating. The coating can also be a gel coating, and extrusion coating, or an abrasion-resistant coating, e.g. for polycarbonate.

The coating composition can be a clear coating, and be transparent to light. The clear coating can be tinted with a dye. The coating composition can also comprise a pigment, and be opaque or translucent. The pigment can be an inorganic pigment, an organic pigment, or a special effect pigment, and can provide an aesthetic effect to the coating film appearance. In some embodiments, the pigment is an inorganic pigment. The inorganic pigment can be, for example, titanium dioxide, zinc oxide, zinc sulfide, lithopone, antimony oxide, iron oxides, iron hydroxides, chromium oxides, chromate pigments, spinel calcined pigments, Prussian Blue, carbon black, or mixtures thereof. In an exemplary embodiment, the metal oxide is titanium dioxide. Titanium dioxide exists in two crystal forms, anatase and rutile, with the rutile form being more photostable. Examples of commercially available rutile titanium dioxide are KRONOS™ 2310, available from Kronos Worldwide, Inc. (Cranbury, N.J.), TI-PURE™ R-900, available from DuPont (Wilmington, Del.), or TiONA™ AT1, commercially available from Millenium Inorganic Chemicals. Titanium dioxide is also available in concentrated dispersion form. An example of a titanium dioxide dispersion is KRONOS™ 4311, also available from Kronos Worldwide, Inc.

In any or all embodiments, the pigment is an organic pigment. The organic pigment can be a polycyclic, azo, or metal complex pigment, e.g. a monoazo, diazo, β-naphthol, naphthol AS, laked azo, benzimidazolone, azo condensation, metal-complex azo, azomethine, isoindolinone, isoindoline, metal complexes such as phthalocyanine, quinacridone, perylene, perinone, indigo, thioindigo, anthraquinone, indanthrone, anthrapyrimidine, flavanthrone, pyranthrone, anthanthrone, dioxazine, triarylcarbonium, quinophthalone, diketopyrrolopyrrole, nitro, quinoline, isoviolanthrone, pteridine and basic dye complex pigments. Specific examples of organic pigments include, for example, C.I. Pigment Red 170, 177, 179, 202, 254, or 264; C.I. Pigment Violet 19 or 23; C.I. Pigment Blue 15, 15:1, 15:2, 15:3, 15:4, 15:6, 16, or 60; C.I. Pigment Yellow 109, 110, 129, 138, 139, 150, or 184; C.I. Pigment Green 7 or 36; C.I. Pigment Orange 48 or 73; a diketo-pyrrolo-pyrrol pigment, a quinacridone pigment, a quinacridone/diketo-pyrrolo-pyrrol pigment, or mixtures thereof “C.I.” refers to “The Color Index”, which is edited by the Society of Dyers and Colourists and the American Association of Textile Chemists and Colorists. The pigment can be a solid solution pigment or a mixture of crystal combinations of pigments.

Pigments can serve other purposes besides providing opacity and color. Pigments can be, for example, (aesthetic) effect pigments, anti-corrosion, magnetically conductive, magnetically shielding, electrically conductive, fluorescent, or phosphorescent. Effect pigments include, for example, color pigments, pearlescent pigments, interference pigments, metallic effect pigments, liquid crystal pigments, and platelet-shaped effect pigments based on iron oxide. Pearlescent pigments have a layered structure so that different wavelengths of light are reflected at different levels. The reflected waves interfere with each other, causing amplification or cancellation. Pearlescent pigments create brilliant interference colors which depend on the viewing angle. Metallic effect pigments comprise metallic flakes, and can be, for example, aluminum, aluminum bronzes, and steel bronzes.

Extenders (fillers) can also be used. The extenders may optionally be added along with the opacifying agent particles. They are added as part of the first portion of the first additive composition. They are naturally occurring minerals and synthetic inorganic pigments that are relatively colorless in coatings. They can be used in combination with more expensive opacifying agent particles such as, for example, metal oxides, to reduce raw material costs. Suitable extenders are nepheline syenite, (25% nepheline, 55% sodium feldspar, and 20% potassium feldspar), feldspar (an aluminosilicate), diatomaceous earth, calcined diatomaceous earth, calcium carbonate, talc (hydrated magnesium silicate), aluminosilicates, silica (silicon dioxide), alumina (aluminum oxide), clay, (hydrated aluminum silicate), kaolin (kaolinite, China clay, hydrated aluminum silicate), mica (hydrous aluminum potassium silicate), pyrophyllite (aluminum silicate hydroxide), perlite, baryte (barium sulfate), wollastonite (calcium metasilicate), dolomite (calcium magnesium carbonate), and mixtures thereof. In an exemplary embodiment, the extenders that can be used include nepheline syenite, calcined diatomaceous earth, or the like, or combinations comprising one or more of the foregoing extenders. The extenders have average particle sizes of about 0.001 to about 1000 μm, specifically about 0.01 to about 500 μm, and more specifically about 1 to about 100 μm. In another embodiment, the extenders have a surface area of about 0.1 to about 200 m²/g, specifically about 2 to about 100 m²/g and more specifically about 5 to about 50 m²/g. The extenders, when present, are generally used in amounts of about 0.1 to about 50 wt. %, specifically about 0.5 to about 20 wt. %, and more specifically about 1 to about 12 wt. %, based on the total weight of the coating composition.

A coated article formed by a method comprises: applying a layer of the coating composition to a surface of a substrate; and drying or allowing the coating composition to dry into a solid film. The “drying” or “allowing the coating composition to dry” step includes, for example, curing, or crosslinking, the coating composition by any suitable means. Application of the coating composition to the substrate can be done by known methods, e.g. by spraying, brushing, rolling, roller coating, pouring, dip coating, spin coating, laminating, injection back molding, coextruding, troweling, knife coating, or for powder coating, electrostatically. Coating thicknesses can be about 3 to 1000 g/m², preferably 10 to 200 g/m². Curing can be by thermal curing, moisture curing, chemical curing, oxidative curing, or actinic radiation curing, e.g. UV curing.

A method of making a stabilized coating film as described herein comprises: adding an effective amount of a stabilizer comprising at least one of a UV absorber, a hindered amine light stabilizer (HALS), or an inorganic UV blocker to a coating composition; applying a layer of the coating composition to a surface of a substrate; and drying or allowing the coating composition to dry to provide the stabilized coating film. The drying or allowing the coating composition to dry step can, optionally, further include curing, or crosslinking, the coating composition. The effective amount of the stabilizer composition can be 0.005 to 10 wt. %, preferably 0.01 to 5 wt. %, and more preferably 0.1 to 2 wt. %, based on the total solids content of the polymeric binder.

A method of stabilizing a coating composition against the deleterious effects of UV-C (190-280 nm) light, includes adding to the coating composition an effective amount of stabilizer composition comprising at least one of a UV absorber, a hindered amine light stabilizer (HALS), or an inorganic UV blocker, wherein the coating composition is more resistant to discoloration when exposed to UV-C (190-280 nm) light compared to the coating composition in the absence of the stabilizer. The effective amount of the stabilizer composition can be 0.005 to 10 wt. %, preferably 0.01 to 5 wt. %, and more preferably 0.1 to 2 wt. %, based on the total solids content of the polymeric binder.

### EXAMPLES

The following examples are provided to assist one skilled in the art to further understand various embodiments contemplated by the description provided herewith. These examples are intended for illustration purposes only and are not to be construed as limiting the scope of the disclosure or claims.

The efficacy of individual stabilizers as well as combinations of stabilizers in protecting coating compositions against discoloration caused by UV-C light was evaluated. VARATHANE™ Ultimate Crystal Clear Polyurethane paint (water-based, 27.4% solids by weight, Interior, Satin, with no stabilizers) and Dow AVANSE™ ST-410 acrylic emulsion (37% solids by weight, with no stabilizers) were the polymeric binders used in the weathering studies in the examples.

The chemical names, commercial names, and suppliers of the additives used are listed in Table 1. TINUVIN™ 1130 (liquid at room temperature, 100 wt. % active ingredient), TINUVIN™ 292 (liquid at room temperature, 100 wt. % active ingredient), and RHODIGARD™ W185 (colloidal dispersion in water, 18.5 wt. % of active ingredient) can be directly added to the coating compositions. Since the polymeric binders were water-based, stabilizers which are solid at room temperature were first dispersed in water with emulsifier (STEPANTEX™ CO-30, Examples 1-7) before adding to the polymeric binders. Combinations of stabilizers were premixed (as described in Ex. 8 to 10) before adding to the polymeric binders.

TABLE 1
Chemical names, commercial names and supplier information
for the additives used in UV-C weathering studies.

| Chemical Name | Commercial Name (Source) | Additive Type |
|---|---|---|
| 2-Hydroxy-4-(octyloxy)benzophenone | CYASORB ™ UV-531 | UV |
| | (Solvay) | Absorber |
| 4-Hydroxybenzophenone | (Sigma Aldrich) | UV |
| | | Absorber |
| Mixture of β-[3-(2-H-Benzotriazole-2-yl)-4-hydroxy-5- | TINUVIN ™ 1130 (BASF) | UV |
| tert.butylphenyl]-propionic acid-poly(ethylene glycol) | | Absorber |
| 300 ester and bis{β-[3-(2-H-Benzotriazole-2-yl)-4- | | |
| hydroxy-5-tert.butylphenyl]-propionic acid} | | |
| poly(ethylene glycol) 300 ester | | |
| 2-(4,6-Bis-(2,4-dimethylphenyl)- | CYASORB ™ UV-1164G | UV |
| 1,3,5-triazin-2-yl)-5-(octyloxy)phenol | (Solvay) | Absorber |
| 85% solution of mixture of 2-[4-[(2-Hydroxy-3- | TINUVIN ™ 400 (BASF) | UV |
| dodecyloxypropyl)oxy]-2-hydroxyphenyl]-4,6-bis(2,4- | | Absorber |
| dimethylphenyl)-1,3,5-triazine and 2-[4-[(2-Hydroxy-3- | | |
| tridecyloxypropyl)oxy]-2-hydroxyphenyl]-4,6-bis(2,4- | | |
| dimethylphenyl)-1,3,5-triazine in 1-methoxy-2-propanol | | |
| Mixture of Bis(1,2,2,6,6-pentamethyl-4-piperidyl) | TINUVIN ™ 292 (BASF) | HALS |
| sebacate & Methyl 1,2,2,6,6-pentamethyl-4-piperidyl | | |
| sebacate | | |
| 2,2,6,6-Tetramethyl-4-piperidinyl stearate (fatty acids | CYASORB ™ UV-3853 | HALS |
| mixture) | (Solvay) | |
| Cerium(IV) oxide | RHODIGARD ™ W185 | Inorganic |
| | (18.5 wt. %, Solvay) | UV Blocker |
| Ethoxylated castor oil | STEPANTEX ™ CO-30 | Dispersant |
| | (Stepan) | |

### Example 1—Preparation of Dispersion of CYASORB™ UV-531

6 g CYASORB™ UV-531 and 1.5 g STEPANTEX™ CO-30 are mixed together in one glass vial at 90° C. to prepare a homogenous mixture. Then 5 g of the hot mixture was added drop wise to a glass vial with 5 g of hot water at 90° C. under agitation at 700 rpm, providing a slightly yellow aqueous dispersion. Afterwards, the prepared dispersion of CYASORB™ UV-531 was cooled to room temperature. The concentration of stabilizer in the aqueous dispersion on a solids basis was 40 wt. %.

### Example 2—Preparation of Dispersion of 4-Hydroxybenzophenone

6 g 4-Hydroxybenzophenone and 1.5 g STEPANTEX CO-30 were mixed together in a glass vial at 90° C. to prepare a homogenous mixture. Then 5 g of the hot mixture was added drop-wise to a glass vial with 5 g of hot water at 90° C. under agitation at 700 rpm, providing a slightly yellow aqueous dispersion. Afterwards, the aqueous dispersion of 4-hydroxybenzophenone was cooled to room temperature. The concentration of stabilizer in the aqueous dispersion on a solids basis was 40 wt. %.

### Example 3—Preparation of Dispersion of CYASORB™ UV-1164G

6 g CYASORB™ UV-1164G and 1.5 g STEPANTEX™ CO-30 were mixed together in a glass vial at 90° C. to prepare a homogenous mixture. Then 5 g of the hot mixture was added drop-wise to a glass vial with 5 g of hot water at 90° C. under agitation at 700 rpm, providing a slightly yellow aqueous dispersion. Afterwards, the aqueous dispersion of CYASORB™ UV-1164G was cooled to room temperature. The concentration of stabilizer in the dispersion was 40 wt. %.

### Example 4—Preparation of Dispersion of TINUVIN™ 400

6 g TINUVIN™ 400 and 1.5 g STEPANTEX CO-30 were mixed together in a glass vial at 90° C. to prepare a homogenous mixture. Then 5 g of the hot mixture was added drop-wise to a glass vial with 5 g of hot water at 90° C. under agitation at 700 rpm, providing a slightly yellow dispersion. Afterwards, the aqueous dispersion of TINUVIN™ 400 was cooled to room temperature. The concentration of stabilizer in the dispersion was 40 wt. %.

### Example 5—Preparation of Dispersion of CYASORB™ UV-3853

6 g CYASORB™ UV-3853 and 1.5 g STEPANTEX™ CO-30 were mixed together in a glass vial at 90° C. to prepare a homogenous mixture. Then 5 g of the hot mixture was added drop-wise to a glass vial with 5 g of hot water at 90° C. under agitation at 700 rpm, providing a white dispersion. Afterwards, the aqueous dispersion of CYASORB™ UV-3853 was cooled to room temperature. The concentration of stabilizer in the dispersion was 40 wt. %.

### Example 6—Preparation of Dispersion of CYASORB™ UV-1164G & CYASORB™ UV-3853

4 g CYASORB™ UV-1164G, 2 g CYASORB™ UV-3853 and 1.5 g STEPANTEX™ CO-30 were mixed together in one glass vial at 90° C. to prepare a homogenous mixture. Then 5 g of the hot mixture was added drop-wise to a glass vial with 5 g of hot water at 90° C. under agitation at 700 rpm, providing a white dispersion. Afterwards, the aqueous dispersion of CYASORB™ UV-1164G and CYASORB™ UV-3853 was cooled to room temperature. The concentration of stabilizer in the dispersion was 40 wt. %.

### Example 7—Preparation of Dispersion of CYASORB™ UV-531 & CYASORB™ UV-3853

4 g CYASORB™ UV-531, 2 g CYASORB™ UV-3853 and 1.5 g STEPANTEX™ CO-30 were mixed together in a glass vial at 90° C. to prepare a homogenous mixture. Then 5 g of the hot mixture was added drop-wise to a glass vial with 5 g of hot water at 90° C. under agitation at 700 rpm, providing a white dispersion. Afterwards, the prepared dispersion of CYASORB™ UV-531 and CYASORB™ UV-3853 is cooled to room temperature. The concentration of stabilizer in the dispersion was 40 wt. %.

### Example 8—Preparation of Mixture of TINUVIN™ 1130 & TINUVIN™ 292 (2:1)

4 g TIVINUN™ 1130 and 2 g TIVINUN™ 292 were mixed together in a glass vial at room temperature to prepare a homogenous mixture. The concentration of active stabilizer was 100 wt. %.

### Example 9—Preparation of Mixture of TINUVIN™ 1130 & TINUVIN™ 292 (1:2)

2 g TINUVIN™ 1130 and 4 g TINUVIN™ 292 were mixed together in a glass vial at room temperature to prepare a homogenous mixture. The concentration of active stabilizer was 100 wt. %.

### Example 10—Preparation of Mixture of CYASORB™ UV-1164G & TINUVIN™ 292 (1:2)

2 g CYASORB™ UV-1164G and 4 g TINUVIN™ 292 are mixed together in one glass vial at 90° C. to prepare a homogenous mixture. Afterwards, the prepared mixture of CYASORB™ UV-1164G & TINUVIN™ 292 is cooled to room temperature. The concentration of active stabilizer is 100 wt. %.

### Example 11—Preparation of Paints with Stabilizers

The general procedure for the preparation of paints containing stabilizers is as follows: Stabilizers (neat, as dispersions in water, or as mixtures) are added drop-wise to 40 g VARATHANE™ Ultimate Polyurethane paint (Water-based, Interior, Crystal Clear, Satin) or 20 g of Dow AVANSE™ ST-410 acrylic emulsion. Then the mixtures are agitated at 400 rpm for 2 hr. The amounts of stabilizers and paint matrix used are listed in Tables 2 to 4.

TABLE 2
Paint samples prepared with 40 g VARATHANE ™
Ultimate Polyurethane paint and stabilizers.

| Paint | |
|---|---|
| Sample | Additive |
| 1-1 | None |
| 1-2 | 0.12 g STEPANTEX ™ CO-30 |
| 1-3 | 0.3 g Ex. 1 |
| 1-4 | 0.3 g Ex. 2 |
| 1-5 | 0.3 g Ex. 3 |
| 1-6 | 0.3 g Ex. 4 |
| 1-7 | 0.3 g Ex. 5 |
| 1-8 | 0.3 g Ex. 6 |
| 1-9 | 0.12 g TINUVIN ™ 1130 |
| 1-10 | 0.12 g TINUVIN ™ 292 |
| 1-11 | 0.12 g Ex. 8 |
| 1-12 | 0.12 g Ex. 9 |
| 1-13 | 0.12 g Ex. 10 |

TABLE 3
Paint samples prepared with 40 g VARATHANE ™ Ultimate
Polyurethane paint and stabilizers

| Paint | |
|---|---|
| Sample | Additives |
| 2-1 | None |
| 2-2 | 0.3 g Ex. 1 |
| 2-3 | 0.3 g Ex. 3 |
| 2-4 | 0.3 g Ex. 4 |
| 2-5 | 0.12 g Ex. 8 |
| 2-6 | 0.649 g RHODIGARD ™ W185 (0.12 g active) |
| 2-7 | 0.649 g RHODIGARD ™ W185 (0.12 g active) and |
| | 0.12 g TINUVIN ™ 1130 |
| 2-8 | 0.649 g RHODIGARD ™ W185 (0.12 g active) and |
| | 0.12 g TINUVIN ™ 292 |
| 2-9 | 0.649 g RHODIGARD ™ W185 (0.12 g active) and |
| | 0.12 g TINUVIN ™ 1130 & TINUVIN ™ 292 (2:1) |

TABLE 4
Paint samples prepared with 20 g Dow AVANSE ™ ST-
410 acrylic emulsion and additives

| Paint Sample | Additives |
|---|---|
| 3-1 | None |
| 3-2 | 0.222 g Ex. 1 |
| 3-3 | 0.222 g Ex. 3 |
| 3-4 | 0.222 g Ex. 5 |
| 3-5 | 0.222 g Ex. 7 |
| 3-6 | 0.089 g TINUVIN ™ 1130 and |
| | TINUVIN ™ 292 (2:1) |
| 3-7 | 0.48 g RHODIGARD ™ W185 |

### Example 12—Preparation of Coatings

The general procedure for the preparation of coatings is as follows: first, place a bare aluminum panel substrate (QLab A24, 4 in.×2 in.) to be coated on a flat surface. Place a bird-type single bar film applicator (BYK 5550, 6 mils gap clearance and 2 in. film width) on the substrate. Pour paint samples (in Table 2, Table 3, and Table 4) in front of the gap of the film applicator in the pulling direction. Afterwards, pull the film applicator at a uniform speed (ca. 25 mm/s). The film applicator is cleaned with acetone immediately and dried before next use. The coating samples are left on a flat surface to dry for 7 days before weathering study. The coating samples prepared and the corresponding paint samples are listed in Table 5.

TABLE 5
Coating samples prepared from corresponding paint samples on bare
aluminum panel substrates using a single bar film applicator.

| Coating | Corresponding |
|---|---|
| Sample | Paint Sample |
| 1-1 | 1-1 |
| 1-2 | 1-2 |
| 1-3 | 1-3 |
| 1-4 | 1-4 |
| 1-5 | 1-5 |
| 1-6 | 1-6 |
| 1-7 | 1-7 |
| 1-8 | 1-8 |
| 1-9 | 1-9 |
| 1-10 | 1-10 |
| 1-11 | 1-11 |
| 1-12 | 1-12 |
| 1-13 | 1-13 |
| 2-1 | 2-1 |
| 2-2 | 2-2 |
| 2-3 | 2-3 |
| 2-4 | 2-4 |
| 2-5 | 2-5 |
| 2-6 | 2-6 |
| 2-7 | 2-7 |
| 2-8 | 2-8 |
| 2-9 | 2-9 |
| 3-1 | 3-1 |
| 3-2 | 3-2 |
| 3-3 | 3-3 |
| 3-4 | 3-4 |
| 3-5 | 3-5 |
| 3-6 | 3-6 |
| 3-7 | 3-7 |

### Example 13—UV-C Weathering of Coating Samples

For UV-C weathering studies, an UV-C weathering device was developed and assembled in-house. It includes two low-pressure, narrow-band UV-C lamps (254 nm) with an average irradiance level of ca. 1200 μW/cm² (at 254 nm) at the plaque surface as well as an automatic fan controller in order to maintain the testing temperature below 40° C. During the UV-C weathering tests, coating samples were placed inside the machine and repositioned frequently to ensure all samples received an equal amount of radiant exposure.

Changes in color (delta E or ΔE) and in Yellowness Index (delta YI or ΔYI) of the coating film after UV-C exposure are used to measure discoloration. Both measurements are conducted using an X-Rite Color i7 spectrophotometer using CIELAB color scale (for color measurement, reporting L* as the lightness coordinate, a* as the red/green coordinate, and b* as the yellow/blue coordinate) and ASTM E303 (for YI measurement), respectively. ΔL*, Δa*, Δb*, and ΔYI are calculated by subtracting the initial data (time=0 hour) from the one after×hours of UV-C exposure. ΔE is calculated with the equation:

ΔE=\sqrt{(ΔL*)²+(Δa*)²+(Δb*)²}

The higher the ΔE, the more the color change or discoloration of sample from the initial state. The higher the ΔYI, the more yellow the samples become. The results of UV-C weathering of the coating samples are listed in Table 6, Table 7, and Table 8.

TABLE 6
Performance of coating samples (prepared
from VARATHANE ™ Ultimate Polyurethane
paint and stabilizers) after exposure
to UV-C weathering for 190 hr.

| Coating | | ΔE | ΔYI |
|---|---|---|---|
| Sample | Stabilizer | (190 hr.) | (190 hr.) |
| 1-1 | None | 9.90 | 17.82 |
| 1-2 | None^{a} | 10.08 | 18.08 |
| 1-3 | 0.3 wt. % CYASORB ™ UV-531^{b} | 4.74 | 8.39 |
| 1-4 | 0.3 wt. % 4-hydroxybenzophenone^{b} | 6.81 | 12.17 |
| 1-5 | 0.3 wt. % CYASORB ™ UV-1164G^{b} | 5.63 | 9.97 |
| 1-6 | 0.3 wt. % TINUVIN ™ 400^{b} | 5.46 | 9.69 |
| 1-7 | 0.3 wt. % CYASORB ™ UV-3853^{b} | 8.55 | 15.29 |
| 1-8 | 0.2 wt. % CYASORB ™ UV-1164G | 6.53 | 11.77 |
| | and 0.1 wt. % CYASORB ™ | | |
| | UV-3853^{b} | | |
| 1-9 | 0.3 wt. % TINUVIN ™ 1130 | 7.71 | 13.95 |
| 1-10 | 0.3 wt. % TINUVIN ™ 292 | 8.12 | 14.49 |
| 1-11 | 0.2 wt. % TINUVIN ™ 1130 and | 7.47 | 13.44 |
| | 0.1 wt. % TINUVIN ™ 292 | | |
| 1-12 | 0.1 wt. % TINUVIN ™ 1130 and | 6.05 | 10.72 |
| | 0.2 wt. % TINUVIN ™ 292 | | |
| 1-13 | 0.1 wt. % CYASORB ™ UV-1164G | 7.49 | 13.4 |
| | and 0.2 wt. % TINUVIN ™ | | |
| | UV-3853 | | |

^{a}Contains 0.3 wt. % STEPANTEX ™ CO-30 emulsifier.
^{b}Contains 0.075 wt. % STEPANTEX ™ CO-30 emulsifier.

TABLE 7
Performance of coating samples (prepared
from VARATHANE ™ Ultimate Polyurethane
paint and stabilizers) after exposure
to UV-C weathering for 216 hr.

| Coating | | ΔE | ΔYI |
|---|---|---|---|
| Sample | Stabilizer | (216 hr.) | (216 hr.) |
| 2-1 | None | 11.63 | 20.75 |
| 2-2 | 0.3 wt. % CYASORB ™ UV-531^{a} | 5.61 | 9.94 |
| 2-3 | 0.3 wt. % CYASORB ™ UV-1164G^{a} | 6.57 | 11.67 |
| 2-4 | 0.3 wt. % TINUVIN ™ 400^{a} | 5.58 | 9.86 |
| 2-5 | 0.2 wt. % TINUVIN ™ 1130 and | 6.28 | 11.14 |
| | 0.1 wt. % TINUVIN ™ 292 | | |
| 2-6 | 0.3 wt. % RHODIGARD ™ W185 | 7.70 | 14.13 |
| 2-7 | 0.3 wt. % RHODIGARD ™ W185 | 6.78 | 12.59 |
| | 0.3 wt. % TINUVIN ™ 1130 | | |
| 2-8 | 0.3 wt. % RHODIGARD ™ W185 | 6.71 | 12.17 |
| | 0.3 wt. % TINUVIN ™ 292 | | |
| 2-9 | 0.3 wt. % RHODIGARD ™ W185 | 6.48 | 11.97 |
| | 0.2 wt. % TINUVIN ™ 1130 and | | |
| | 0.1 wt. % TINUVIN ™ 292 | | |

^{a}Contains 0.075 wt. % STEPANTEX ™ CO-30 emulsifier.

TABLE 8
Performance of coating samples (prepared with Dow
AVANSE ™ ST-410 acrylic emulsion and stabilizers)
after exposure to UV-C weathering for 270 hours.

| Coating | | ΔE | ΔYI |
|---|---|---|---|
| Sample | Stabilizer | (270 hr.) | (270 hr.) |
| 3-1 | None | 5.71 | 9.69 |
| 3-2 | 0.44 wt. % CYASORB ™ UV-531^{a} | 2.41 | 3.99 |
| 3-3 | 0.44 wt. % CYASORB ™ UV-1164G^{a} | 2.26 | 3.61 |
| 3-4 | 0.44 wt. % CYASORB ™ UV-3853^{a} | 3.69 | 6.16 |
| 3-5 | 0.29 wt. % CYASORB ™ UV-531 | 2.92 | 4.85 |
| | 0.15 wt. % CYASORB ™ UV-3853^{a} | | |
| 3-6 | 0.29 wt. % TINUVIN ™ 1130 | 3.50 | 5.84 |
| | 0.15 wt. % TINUVIN ™ 292 | | |
| 3-7 | 0.44 wt. % RHODIGARD ™ W185 | 0.40 | 0.42 |

^{a}Contains 0.11 wt. % STEPANTEX ™ CO-30 emulsifier.

The data in Table 6 and Table 7 demonstrate that polymeric binder VARATHANE™ Ultimate Crystal Clear Polyurethane (Coating Sample 1-1 and Coating Sample 2-1) suffers from significant discoloration (as evidenced by ΔE and ΔYI results) after UV-C exposure of 190 hr. and 216 hr. The ΔE and ΔYI results of Coating Sample 1-2 shows that emulsifier itself, i.e., STEPANTEX™ CO-30, has no impact on discoloration compared to the control with no additive (Coating Sample 1-1). The data in Tables 6 and 7 show that stabilizer compositions comprising at least one of UV absorbers, hindered amine light stabilizers (HALS), or inorganic UV blockers, mitigates polymer discoloration after UV-C light exposure. Lower ΔYI and ΔE values were obtained for Coating Samples 1-3 to 1-13 (Table 6) and Coating Samples 2-2 to 2-9 (Table 7). The best results (lowest color change and discoloration),were obtained with CYASORB™ UV-531 (Coating Samples 1-2 and 2-2) and TINUVIN™ 400 (Coating Sample 2-4). The CYASORB™ UV-531 results are surprising, because it belongs to the genus of ortho-hydroxyl benzophenones, which are generally considered in the art as inferior stabilizers for protection against UV-A and UV-B light exposure.

Similar results were obtained with coating compositions formulated with Dow AVANSE™ ST-410 acrylic emulsion as the polymeric binder. The results are summarized in Table 8. Although the amount of discoloration was not as severe as with VARATHANE™ polyurethane, AVANSE™ ST-410 acrylic (Coating Sample 3-1) still suffers from significant discoloration suffers from significant discoloration (as evidenced by ΔE and ΔYI results) after UV-C exposure of 270 hr.

The data in Table 8 show that stabilizer compositions comprising at least one of UV absorbers, hindered amine light stabilizers (HALS), or inorganic UV blockers, mitigate polymer discoloration upon UV-C light exposure. Lower ΔYI and ΔE values were obtained for Coating Samples 3-2 to 3-7. RHODIGARD™ W185 performed extremely well, indicating that cerium(IV) oxide (an inorganic UV blocker) is highly effective in reducing discoloration of coatings after UV-C exposure. CYASORB™ UV-531, CYASORB™ UV-1164G, and a mixture of CYASORB™ UV-531 and CYASORB™ UV-3853 were also effective in reducing discoloration upon UV-C exposure.

Taken together, these results indicate that stabilizer compositions comprising at least one of a UV absorber, hindered amine light stabilizer (HALS), inorganic UV blocker, improve resistance of a coating composition to the adverse effects (e.g. discoloration) of exposure to UV-C light.

Various patent and/or scientific literature references have been referred to throughout this application. The disclosures of these publications in their entireties are hereby incorporated by reference as if written herein. In view of the above description and the examples, one of ordinary skill in the art will be able to practice the disclosure as claimed without undue experimentation.

Although the foregoing description has shown, described, and pointed out the fundamental novel features of the present teachings, it will be understood that various omissions, substitutions, and changes in the form of the details of the coating compositions and method as illustrated, as well as the uses thereof, may be made by those skilled in the art, without departing from the scope of the present teachings.

We claim:

1. A coating composition comprising
a polymeric binder and
a stabilizer composition comprising at least one of a UV absorber, a hindered amine light stabilizer (HALS), or an inorganic UV blocker,
wherein the coating composition is more resistant to discoloration when exposed to UV-C (190-280 nm) light compared to the coating composition in the absence of the stabilizer composition.

2. The coating composition of claim 1, wherein the stabilizer composition is present in an amount of 0.005 to 10 wt. %, preferably 0.01 to 5 wt. %, and more preferably 0.1 to 2 wt. %, based on the total solids content of the polymeric binder.

3. The coating composition of claim 1, wherein the stabilizer composition comprises a UV absorber.

4. The coating composition of claim 3, wherein the UV absorber comprises at least one of a 2-(2′-hydroxyphenyl)-s-triazine, 2-hydroxybenzophenone, 2-(2′-hydroxyphenyl)benzotriazole, or benzoxazinone.

5. The coating composition of claim 3, wherein the UV absorber comprises at least one 2-(2′-hydroxyphenyl)-s-triazine according to Formula (I): 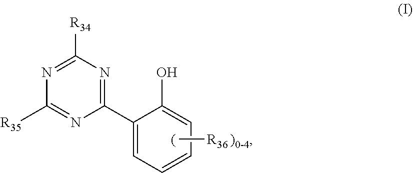
wherein
each of R₃₄ and R₃₅ is independently chosen from a C₆-C₁₀ aryl group, mono- or di-C₁-C₁₂ hydrocarbyl-substituted amino, C₂-C₁₂ alkanoyl, C₁-C₁₂ alkyl, C₁-C₁₀ acyl, or C₁-C₁₀ alkoxyl,
wherein the C₆-C₁₀ aryl group is optionally substituted at from 1 to 3 substitutable positions with at least one of OH, halogen, C₁-C₁₂ alkyl, C₁-C₁₂ alkoxy, C₁₋₁₂ alkoxyester, C₂₋₁₂ alkanoyl, or phenyl, wherein the phenyl is optionally substituted at from 1 to 3 substitutable positions with at least one of OH, halogen, C₁₋₁₂ alkyl, C₁₋₁₂ alkoxy, C₁₋₁₂ alkoxyester, or C₂₋₁₂ alkanoyl; and
each instance of R₃₆ is independently chosen from OH, halogen, C₁-C₁₂ alkyl, C₁-C₁₂ alkoxy, C₁-C₁₂ alkoxyester, C₂-C₁₂ alkanoyl, phenyl, or C₁-C₁₂ acyl.

6. The coating composition of claim 5, wherein the 2-(2′-hydroxyphenyl)-s-triazine comprises at least one of:
4,6-diphenyl-2-(4-hexyloxy-2-hydroxyphenyl)-s-triazine (TINUVIN™ 1577),
4,6-bis-(2,4-dimethylphenyl)-2-(2-hydroxy-4-octyloxyphenyl)-s-triazine (CYASORB™ UV-1164),
4,6-bis-(2,4-dimethylphenyl)-2-(2-hydroxy-4-isooctyloxyphenyl)-s-triazine (CYASORB™ UV-1164L),
2,4-bis[2-hydroxy-4-(2-hydroxy-4-(2-hydroxyethoxy)phenyl]-6-(2,4-dimethylphenyl)-s-triazine,
mixture of 4,6-bis(2,4-dimethylphenyl)-2-(2-hydroxy-4-(3-dodecyloxy-2-hydroxypropoxy)phenyl)-s-triazine and 4,6-bis-(2,4-dimethylphenyl)-2-(2-hydroxy-4-(3-tridecyloxy-2-hydroxypropoxy)phenyl)-s-triazine (TINUVIN™ 400),
4,6-bis(2,4-dimethylphenyl)-2-(2-hydroxy-4(3-(2-ethylhexyloxy)-2-hydroxypropoxy)-phenyl)-s-triazine (TINUVIN™ 405),
4,6-bis(2,4-dimethylphenyl)-2-(2-hydroxy-4(3-(2-ethylhexyloxy)-2-hydroxypropoxy)-phenyl)-s-triazine,
2,4-bis(2,4-dimethylphenyl)-6-[2-hydroxy-4-[(octyloxycarbonyl)ethylideneoxy]phenyl]-s-triazine (TINUVIN™ 479),
2,4-bis(4-biphenylyl)-6-[2-hydroxy-4-[(octyloxycarbonyl)ethylideneoxy]phenyl]-s-triazine,
2,4-bis(4-biphenylyl)-6-[2-hydroxy-4-(2-ethylhexyloxy)phenyl]-s-triazine (TINUVIN™ 1600),
2,4-bis(2-hydroxy-4-butyloxyphenyl)-6-(2,4-bis-butyloxyphenyl)-s-triazine (TRIAZINE™ 460),
2,4,6-tris[2-hydroxy-4-(3-sec-butyloxy-2-hydroxypropyloxy)-phenyl]-s-triazine, or
2,4,6-tris[2-hydroxy-4-[(octyloxycarbonyl)ethylideneoxy]phenyl]-s-triazine (TINUVIN™ 477).

7. The coating composition of claim 3, wherein the UV absorber comprises a 2-hydroxybenzophenone chosen from at least one of 2-hydroxy-4-methoxybenzophenone (CYASORB™ UV-9), 2,2′-dihydroxy-4-methoxybenzophenone (CYASORB™ UV-24), 2-hydroxy-4-octyloxybenzophenone (CYASORB™ UV-531), 2,2′-dihydroxy-4,4′-di-methoxybenzophenone, 2,2′-dihydroxybenzophenone, 2,2′,4,4′-tetrahydroxybenzophenone, 2,2′-dihydroxy-4,4′-dimethoxybenzophenone, 2,2′-dihydroxy-4,4′-diethoxybenzophenone, 2,2′-dihydroxy-4,4′-dipropoxybenzophenone, 2,2′-dihydroxy-4,4′-dibutoxybenzophenone, 2,2′-dihydroxy-4-methoxy-4′-ethoxybenzophenone, 2,2′-dihydroxy-4-methoxy-4′-propoxybenzophenone, 2,2′-dihydroxy-4-methoxy-4′-butoxybenzophenone, 2,2′-dihydroxy-4-ethoxy-4′-propoxybenzophenone, 2,2′-dihydroxy-4-ethoxy-4′-butoxybenzophenone, 2,3′-dihydroxy-4,4′-dimethoxybenzophenone, 2,3′-dihydroxy-4-methoxy-4′-butoxybenzophenone, 2-hydroxy-4,4′,5′-trimethoxybenzophenone, 2-hydroxy-4,4′,6′-tributoxybenzophenone, 2-hydroxy-4-butoxy-4′,5′-dimethoxybenzophenone, 2-hydroxy-4-ethoxy-2′,4′-dibutylbenzophenone, 2-hydroxy-4-propoxy-4′,6′-dichlorobenzophenone, 2-hydroxy-4-propoxy-4′,6′-dibromobenzophenone, 2,4-dihydroxybenzophenone, 2-hydroxy-4-ethoxybenzophenone, 2-hydroxy-4-propoxybenzophenone, 2-hydroxy-4-butoxybenzophenone, 2-hydroxy-4-methoxy-4′-methylbenzophenone, 2-hydroxy-4-methoxy-4′-ethylbenzophenone, 2-hydroxy-4-methoxy-4′-propylbenzophenone, 2-hydroxy-4-methoxy-4′-butylbenzophenone, 2-hydroxy-4-methoxy-4′-tert-butylbenzophenone, 2-hydroxy-4-methoxy-4′-chlorobenzophenone, 2-hydroxy-4-methoxy-2′-chlorobenzophenone, 2-hydroxy-4-methoxy-4′-bromobenzophenone, 2-hydroxy-4,4′-dimethoxybenzophenone, 2-hydroxy-4,4′-dimethoxy-3-methylbenzophenone, 2-hydroxy-4,4′-dimethoxy-2′-ethylbenzophenone, 2-hydroxy-4,4′,5′-trimethoxybenzophenone, 2-hydroxy-4-ethoxy-4′-methylbenzophenone, 2-hydroxy-4-ethoxy-4′-ethylbenzophenone, 2-hydroxy-4-ethoxy-4′-propylbenzophenone, 2-hydroxy-4-ethoxy-4′-butylbenzophenone, 2-hydroxy-4-ethoxy-4′-methoxybenzophenone, 2-hydroxy-4,4′-diethoxybenzophenone, 2-hydroxy-4-ethoxy-4′-propoxybenzophenone, 2-hydroxy-4-ethoxy-4′-butoxybenzophenone, 2-hydroxy-4-ethoxy-4′-chlorobenzophenone, or 2-hydroxy-4-ethoxy-4′-bromobenzophenone.

8. The coating composition of claim 3, wherein the UV absorber comprises a 2-(2′-hydroxyphenyl)benzotriazole that is at least one of 2-(2′-hydroxy-5′-methylphenyl)benzotriazole (TINUVIN™ P), 2-(2′-hydroxy-5′-tert-butylphenyl)benzotriazole, 2-(2′-hydroxy-3′-methyl-5′-tert-butylphenyl)benzotriazole, 2-(2′-hydroxy-5′-cyclohexylphenyl)benzotriazole, 2-(2′-hydroxy-3′,5′-dimethylphenyl)benzotriazole, 2-(2′-hydroxy-5′-tert-butylphenyl)-5-chloro-benzotriazole, 2-(2′-hydroxy-5′-tert-octylphenyl)benzotriazole (CYASORB™ UV-5411), 2-(3′,5′-di-tert-butyl-2′-hydroxyphenyl)-5-chlorobenzotriazole, 2-(3′,5′-di-tert-amyl-2′-hydroxyphenyl)benzotriazole (CYASORB™ UV-2337), 2-(3′,5′-bis(αα-dimethylbenzyl)-2′-hydroxyphenyl)benzotriazole (TINUVIN™ 900), 2-(3′-tert-butyl-2′-hydroxy-5′-(2-octyloxycarbonylethyl)phenyl)benzotriazole, 2,2′-methylenebis[4-(1,1,3,3-tetramethylbutyl)-6-benzotriazole-2-ylphenol], the transesterification product of 2-[3′-tert-butyl-5′-(2-methoxycarbonylethyl)-2′-hydroxyphenyl]-2H-benzotriazole with polyethylene glycol 300 (TINUVIN™ 1130), 2-[2′-hydroxy-3′-(α,α-dimethylbenzyl)-5′-(1,1,3,3-tetramethylbutyl)phenyl]benzotriazole, 5-trifluoromethyl-2-(2-hydroxy-3-α-cumyl-5-tert-octylphenyl)benzotriazole, 2-(2′-hydroxy-5′-(2-hydroxyethyl)phenyl)benzotriazole, 2-(2′-hydroxy-5′-(2-methacryloyloxyethyl)phenyl)benzotriazole, 2-(2′-hydroxy-3′-tert-butyl-5′-methylphenyl)-5-chlorobenzotriazole (TINUVIN™ 326), 2-(3′-sec-butyl-5′-tert-butyl-2′-hydroxyphenyl)benzotriazole, 2-(3′,5′-di-tert-butyl-2′-hydroxyphenyl)benzotriazole, 2-(3′-dodecyl-5′-methyl-2′-hydroxyphenyl)-benzotriazole, 2-(3′-tert-butyl-5′-(2-octyloxycarbonylethyl)-2′-hydroxyphenyl)-5-chlorobenzotriazole, 2-(5′-methyl-2′-hydroxyphenyl)benzotriazole, or 2-(5′-tert-butyl-2′-hydroxyphenyl)benzotriazole.

9. The coating composition of claim 3, wherein the UV absorber comprises a benzoxazinone that is at least one of 2-methyl-3,1-benzoxazin-4-one, 2-butyl-3,1-benzoxazin-4-one, 2-phenyl-3,1-benzoxazin-4-one, 2-(1- or 2-naphthyl)-3,1-benzoxazin-4-one, 2-(4-biphenyl)-3,1-benzoxazin-4-one, 2-(4-biphenyl)-3,1-benzoxazin-4-one, 2-p-nitrophenyl-3,1-benzoxazin-4-one, 2-m-nitrophenyl-3,1-benzoxazin-4-one, 2-p-benzoylphenyl-3,1-benzoxazin-4-one, 2-p-methoxyphenyl-3,1-benzoxazin-4-one, 2-O-methoxyphenyl-3,1-benzoxazin-4-one, 2-cyclohexyl-3,1-benzoxazin-4-one, 2p-(or m-)phthalimidephenyl-3,1-benzoxazin-4-one, N-phenyl-4-(3,1-benzoxazin-4-one-2-yl)phthalimide, N-benzoyl-4-(3,1-benzoxazine-4-one-2-yl)aniline, N-benzoyl-N-methyl-4-(3,1-benzoxazin-4-one-2-yl)-aniline, 2-[p-(N-phenylcarbamonyl)phenyl]-3,1-benzoxazin-4-one, 2-[p-(N-phenyl N-methylcarbamoyl)phenyl]-3,1-benzoxazin-4-one, 2,2′-bis(3,1-benzoxazin-4-one), 2,2′-ethylenebis(3,1-benzoxazin-4-one), 2,2′-tetramethylenebis(3,1-benzoxazin-4-one), 2,2′-hexamethylenebis(3,1-benzoxazin-4-one), 2,2′-decamethylenebis(3,1-benzoxazin-4-one), 2,2′-p-phenylenebis(3,1-benzoxazin-4-one) (CYASORB™ UV-3638), 2,2′-m-phenylenebis(3,1-benzoxazin-4-one), 2,2′-(4,4′-diphenylene)bis(3,1-benzoxazin-4-one), 2,2′-(2,6- or 1,5-naphthalene)bis(3,1-benzoxazin-4-one), 2,2′-(2-methyl-p-phenylene)bis(3,1-benzoxazin-4-one), 2,2′-(2-nitro-p-phenylene)bis(3,1-benzoxazin-4-one), 2,2′-(2-chloro-p-phenylene)bis(3,1-benzoxazin-4-one), 2,2′-(1,4-cyclohexylene)bis(3,1-benzoxazin-4-one), N-p-(3,1-benzoxazin-4-on-2-yl)phenyl, 4-(3,1-benzoxazin-4-on-2-yl)phthalimide, N-p-(3,1-benzoxazin-4-on-2-yl)benzoyl, 4-(3,1-benzoxazin-4-on-2-yl)aniline, 1,3,5-tri(3,1-benzoxazin-4-on-2-yl)benzene, 1,3,5-tri(3,1-benzoxazin-4-on-2-yl)naphthalene, or 2,4,6-tri(3,1-benzoxazin-4-on-2-yl)naphthalene.

10. The coating composition of claim 3, wherein the UV absorber comprises a 2-(2′-hydroxyphenyl)-s-triazine and a 2-(2′-hydroxyphenyl)benzotriazole.

11. The coating composition of claim 1, wherein the UV absorber is at least one of 2-hydroxy-4-octyloxybenzophenone (CYASORB™ UV-531), 4-hydroxybenzophenone, 2-(4,6-bis-(2,4-dimethylphenyl)-1,3,5-triazin-2-yl)-5-(isooctyloxy)phenol (CYASORB™ UV-1164L), mixture of 2-[4-[(2-hydroxy-3-dodecyloxypropyl)oxy]-2-hydroxyphenyl]-4,6-bis(2,4-dimethylphenyl)-1,3,5-triazine and 2-[4-[(2-hydroxy-3-tridecyloxypropyl)oxy]-2-hydroxyphenyl]-4,6-bis(2,4-dimethylphenyl)-1,3,5-triazine in 1-methoxy-2-propanol (TINUVIN™ 400), mixture of β-[3-(2-H-benzotriazole-2-yl)-4-hydroxy-5-tert-butylphenyl]-propionic acid poly(ethylene glycol) 300 ester and bis{β-[3-(2-H-benzotriazole-2-yl)-4-hydroxy-5-tert-butylphenyl]-propionic acid} poly(ethylene glycol) 300 ester (TINUVIN™ 1130).

12. The coating composition of claim 1, wherein the stabilizer composition comprises an inorganic UV blocker.

13. The coating composition of claim 12, wherein the inorganic UV blocker comprises at least one of titanium dioxide, zinc oxide, cerium(IV) oxide, or barium sulfate.

14. The coating composition of claim 1, wherein the stabilizer composition comprises a hindered amine light stabilizer (HALS).

15. The coating composition of claim 14, wherein the HALS comprises
at least one functional group according to Formula (II): 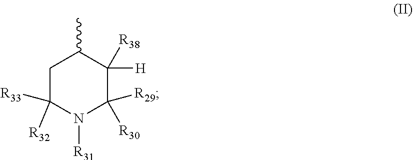
wherein:
R₃₁ is hydrogen, OH, C₁-C₂₀ hydrocarbyl,—CH₂CN, C₁-C₁₂ acyl, or C₁-C₁₈ alkoxy;
R₃₈ is hydrogen or C₁-C₈ hydrocarbyl; and
each of R₂₉, R₃₀, R₃₂, and R₃₃ is independently chosen from C₁-C₂₀ hydrocarbyl, or R₂₉ and R₃₀ and/or R₃₂ and R₃₃ taken together with the carbon to which they are attached form a C₅-C₁₀ cycloalkyl; or
at least one functional group according to Formula (IIa): 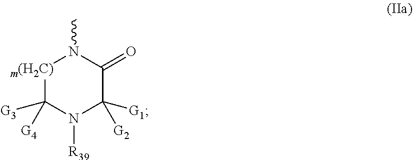
wherein:
m is an integer from 1 to 2;
R₃₉ is hydrogen, OH, C₁-C₂₀ hydrocarbyl,—CH₂CN, C₁-C₁₂ acyl, or C₁-C₁₈ alkoxy; and
each of G₁-G₄ is independently chosen from C₁-C₂₀ hydrocarbyl.

16. The coating composition of claim 14, wherein the hindered amine light stabilizer (HALS) comprises at least one of:
bis(2,2,6,6-tetramethylpiperidin-4-yl) sebacate (TINUVIN™ 770);
bis(2,2,6,6-tetramethylpiperidin-4-yl) succinate;
a mixture of bis(1,2,2,6,6-pentamethylpiperidin-4-yl) sebacate and methyl 1,2,2,6,6-pentamethylpiperidin-4-yl sebacate (TINUVIN™ 292);
bis(1-octyloxy-2,2,6,6-tetramethylpiperidin-4-yl) succinate;
bis(1-octyloxy-2,2,6,6-tetramethylpiperidin-4-yl) sebacate (TINUVIN™ 123);
a mixture of 2,2,6,6-tetramethylpiperidin-4-yl esters of C₁₂-C₂₁ saturated and C₁₈ unsaturated fatty acids (CYASORB™ UV-3853);
bis(1,2,2,6,6-pentamethylpiperidin-4-yl) n-butyl 3,5-di-tert-butyl-4-hydroxybenzylmalonate;
a condensate of 1-(2-hydroxyethyl)-2,2,6,6-tetramethyl-4-hydroxypiperidine and succinic acid (TINUVIN™ 622);
2,2,6,6-tetramethylpiperidin-4-yl stearate;
2,2,6,6-tetramethylpiperidin-4-yl dodecanate;
1,2,2,6,6-pentamethylpiperidin-4-yl stearate;
1,2,2,6,6-pentamethylpiperidin-4-yl dodecanate;
a condensate of N,N′-bis(2,2,6,6-tetramethylpiperidin-4-yl)hexamethylenediamine and 4-tert-octylamino-2,6-dichloro-1,3,5-triazine (CHIMASSORB™ 944);
tris(2,2,6,6-tetramethylpiperidin-4-yl) nitrilotriacetate;
4-stearyloxy-2,2,6,6-tetramethylpiperidine;
a condensate of N,N′-bis(2,2,6,6-tetramethylpiperidin-4-yl)hexamethylenediamine and 4-morpholino-2,6-dichloro-1,3,5-triazine (CYASORB™ UV-3346);
a condensate of N,N′-bis(2,2,6,6-tetramethylpiperidin-4-yl)hexamethylenediamine and 4-morpholino-2,6-dichloro-1,3,5-triazine, methylated (CYASORB™ UV-3529);
a condensate of 2-chloro-4,6-bis(4-n-butylamino-2,2,6,6-tetramethylpiperidyl)-1,3,5-triazine and 1,2-bis(3-aminopropylamino)ethane (CHIMASSORB™ 119);
a condensate of 2-chloro-4,6-bis(4-n-butylamino-1,2,2,6,6-pentamethylpiperidyl)-1,3,5-triazine and 1,2-bis-(3-aminopropylamino)ethane;
a condensate of N,N′-bis(2,2,6,6-tetramethylpiperidin-4-yl)hexamethylenediamine, N-butyl-2,2,6,6-tetramethyl-4-piperidinamine, di-n-butyl amine, and 2,4,6-trichloro-1,3,5-triazine (CHIMASSORB™ 2020);
a mixture of 4-hexadecyloxy- and 4-stearyloxy-1,2,2,6,6-pentamethylpiperidine;
a condensate of N,N′-bis(2,2,6,6-tetramethylpiperidin-4-yl)hexamethylenediamine and 4-cyclohexylamino-2,6-dichloro-1,3,5-triazine;
a condensate of 1,2-bis(3-aminopropylamino)ethane, 2,4,6-trichloro-1,3,5-triazine, and 4-butylamino-2,2,6,6-tetramethylpiperidine;
a condensate of N,N′-bis(2,2,6,6-tetramethylpiperidin-4-yl)hexamethylenediamine and 4-cyclohexylamino-2,6-dichloro-1,3,5-triazine;
tetrakis(2,2,6,6-tetramethylpiperidin-4-yl)-1,2,3,4-butanetetracarboxylate;
tetrakis(1,2,2,6,6-pentamethylpiperidin-4-yl)-1,2,3,4-butanetetracarboxylate;
1,2,3,4-butanetetracarboxylic acid, 2,2,6,6-tetramethylpiperidinyl-4-yl tridecyl ester;
1,2,3,4-butanetetracarboxylic acid, 1,2,2,6,6-pentamethylpiperidin-4-yl tridecyl ester;
formamide, N,N′-1,6-hexanediylbis[N-(2,2,6,6-tetramethylpiperidin-4-yl) (UVINUL™ 4050);
a condensate of N,N′-bis(2,2,6,6-tetramethyl-1-(propyloxy)-piperidin-4-yl)hexamethylenediamine, N-butyl-1-propyloxy-2,2,6,6-tetramethyl-4-piperidinamine, di-n-butyl amine, and 2,4,6-trichloro-1,3,5-triazine (TINUVIN™ NOR HALS 371);
N,N′-bis(2,2,6,6-tetramethyl-4-piperidin-4-yl)hexamethylene diamine, polymer with 2,4,6-trichloro-1,3,5-triazine, reaction products with 3-bromo-1-propene, di-n-butylamine, and N-butyl-2,2,6,6-tetramethyl-4-piperidinamine, oxidized, hydrogenated (TINUVIN™ XT 200);
TINUVIN™ XT-850/XT-855; or
N¹,N¹′-1,2-ethanediylbis(1,3-propanediamine), reaction products with cyclohexane and peroxidized N-butyl-2,2,6,6-tetramethyl-4-piperidinamine-2,4,6-trichloro-1,3,5-triazine (FLAMESTAB™ NOR 116).

17. The coating composition of claim 14, wherein the hindered amine light stabilizer (HALS) is at least one of a mixture of 2,2,6,6-tetramethylpiperidin-4-yl esters of C₁₂-C₂₁ saturated and C₁₈ unsaturated fatty acids (CYASORB™ UV-3853), a mixture of bis(1,2,2,6,6-pentamethylpiperidin-4-yl) sebacate and methyl 1,2,2,6,6-pentamethylpiperidin-4-yl sebacate (TINUVIN™ 292), or bis(1-octyloxy-2,2,6,6-tetramethylpiperidin-4-yl) sebacate (TINUVIN™ 123).

18. The coating composition of claim 1, wherein the stabilizer composition comprises a UV absorber and a hindered amine light stabilizer (HALS).

19. The coating composition of claim 1, wherein the stabilizer composition comprises an inorganic UV blocker in combination with a UV absorber, a hindered amine light stabilizer (HALS), or mixture thereof.

20. The coating composition of claim 1, wherein the coating composition is a solvent-borne coating, a water-borne coating, a plastisol, a powder coating, or a coextruded thermoplastic.

21. The coating composition of claim 1, further comprising a pigment.

22. The coating composition of claim 1, further comprising water as a liquid medium.

23. The coating composition of claim 1, wherein the polymeric binder comprises a polyacrylate, a polyurethane, or mixture thereof.

24. The coating composition of claim 1, wherein the coating composition is a transportation coating, an automotive coating, a refinish coating, a general industrial coating, a coil coating, a heavy-duty maintenance coating, a marine coating, an architectural coating, a wood stain or varnish, a paper coating, an ink, a laminate, an adhesive, a sealant, a liquid gasket, or a knifing filler.

25. A method of forming a coated article, the method comprising:
applying a layer of a coating composition as defined in claim 1 to a surface of a substrate; and
drying or allowing the coating composition to dry by any suitable means.

26. A method of making a stabilized coating film, the method comprising:
adding an effective amount of a stabilizer composition comprising at least one of a UV absorber, a hindered amine light stabilizer (HALS), or an inorganic UV blocker to a coating composition comprising a polymeric binder;
applying a layer of the coating composition to a surface of a substrate; and
drying or allowing the coating composition to dry by any suitable means, thereby providing a stabilized coating film.

27. The method of claim 26, wherein the effective amount of the stabilizer composition is from 0.005 to 10 wt. %, preferably 0.01 to 5 wt. %, and more preferably 0.1 to 2 wt. %, based on the total solids content of the polymeric binder.

28. The method of claim 26, wherein the stabilized coating film is more resistant to discoloration when exposed to UV-C (190-280 nm) light compared to the coating film in the absence of the stabilizer composition.

29. A method of stabilizing a coating composition against the deleterious effects of UV-C (190-280 nm) light, the method comprising
adding to the coating composition comprising a polymeric binder, an effective amount of stabilizer composition comprising at least one of a UV absorber, a hindered amine light stabilizer (HALS), or an inorganic UV blocker,
wherein the coating composition is more resistant to discoloration when exposed to UV-C (190-280 nm) light compared to the coating composition in the absence of the stabilizer composition.

30. The method of claim 29, wherein the effective amount of the stabilizer composition is from 0.005 to 10 wt. %, preferably 0.01 to 5 wt. %, and more preferably 0.1 to 2 wt. %, based on the total solids content of the polymeric binder.
